(19) United States
(12) Patent Application Publication
(10) Pub. No.: US 2019/0374417 A1
(43) Pub. Date: Dec. 12, 2019

(54) MEDICAL SYSTEM, TABLE TOP TRANSFER METHOD IN MEDICAL SYSTEM, AND ROBOTIC OPERATION TABLE FOR USE IN MEDICAL SYSTEM

(71) Applicant: MEDICAROID CORPORATION, Kobe-shi (JP)

(72) Inventors: Mitsuichi HIRATSUKA, Kobe-shi (JP); Yoshiyuki TAMURA, Kobe-shi (JP); Tetsuya NAKANISHI, Kobe-shi (JP); Yukihiko KITANO, Kobe-shi (JP); Yutaro YANO, Kobe-shi (JP)

(73) Assignee: MEDICAROID CORPORATION, Kobe-shi (JP)

(21) Appl. No.: 16/551,679

(22) Filed: Aug. 26, 2019

Related U.S. Application Data
(63) Continuation of application No. PCT/JP2018/007078, filed on Feb. 27, 2018.

(30) Foreign Application Priority Data
Feb. 28, 2017 (JP) 2017-037758

(51) Int. Cl. A61G 7/018; A61G 13/04; A61G 13/06; A61G 7/008; A61G 7/012; B25J 9/16

(52) U.S. Cl. CPC A61G 7/018; A61G 13/04; A61G 13/06; A61G 7/008; A61B 34/30; B25J 9/1679; A61G 2210/50; A61G 2203/20; A61G 2203/72; A61G 7/012

(57) ABSTRACT

A medical system according to one or more embodiments may include a memory, a robotic operation table top, and a controller. The robotic operation table includes a table top and a robot arm supporting the table top. The controller gives the robot arm a command to move the table top to positions corresponding to the medical processes. The robot arm includes a plurality of joints, a plurality of movable elements connected by the plurality of joints to one another, and a plurality of electric actuators and a plurality of position detectors. At least one of the plurality of joints is a horizontally-rotating joint which couples two movable elements among the plurality of movable elements to each other so as to be rotatable about a vertical axis. The positions corresponding to the medical processes include a placement position, an imaging position, and a surgery position.

### CROSS-REFERENCE TO RELATED APPLICATION

This application is a continuation of International Application No. PCT/JP2018/007078 filed on Feb. 27, 2018, which claims priority to Japanese Patent Application No. 2017-037758 filed on Feb. 28, 2017. The entire contents of these applications are incorporated herein by reference.

### TECHNICAL FIELD

One or more embodiments disclosed herein relate to: a medical system which performs transfer of a patient associated with medical processes such as placement of the patient on a table top, surgery, and imaging; a transfer method for the table top in the medical system; and a robotic operation table for use in the medical system.

### BACKGROUND ART

In recent years, a robot arm is used to support a table top on which a patient is to be placed. The robot arm is also used to determine the position of the patient and transport the patient in the course of surgery or treatment. Japanese Unexamined Patent Publication No. 2009-131718 discloses that robot arms are used in the field of radiotherapy as a means for determining the position of a treatment table. European Patent Publication No. 1985237 discloses that patient positioning robotic systems are also used as a means for transferring the treatment table to a medical imaging device, such as an angiographic device. European Patent Publication No. 2135554 discloses that there are also systems that allow a robot to move the table top, on which a patient lies, while both surgery and medical imaging are carried out.

### SUMRIARY

Such positioning devices or the like which utilize a robot have contributed to increasing, for example, a range of movement and patient transfer efficiency. It may be preferable, however, to take into account an increase in overall efficiency from preparation to completion of a medical process and an improvement in the utilization rate of a medical room as well.

An object of one or more embodiments may be to increase the flexibility of the positions where the patient is transferred, which is associated with medical processes such as placement of the patient on a table top, surgery, and imaging, and to provide a medical system for efficiently carrying out the medical processes, a transfer method for the table top in the medical system, and a robotic operation table for use in the medical system.

A medical system according to one or more embodiments may include: a memory which stores data relating to medical processes including at least a placement process, an imaging process, and a surgery process; a robotic operation table including a table top on which a patient is to be placed and a robot arm supporting the table top, wherein the robot arm includes: a plurality of movable elements; a plurality of joints connecting the plurality of movable elements to one another; a plurality of electric actuators provided corresponding to the plurality of joints respectively; and a plurality of position detectors provided corresponding to the plurality of joints respectively; and wherein at least one of the plurality of joints is a horizontally-rotating joint which couples two movable elements among the plurality of movable elements to each other so as to be rotatable about a vertical axis; and a controller which gives the robot arm a command, which is based on the data relating to the medical processes stored in the memory, to move the table top to positions corresponding to the medical processes including a placement position where the patient is placed on the table top, an imaging position where an image of the patient is taken by a medical imaging device, and a surgery position where surgery is performed on the patient.

A table top transfer method in a medical system according to one or more embodiments may be a table top transfer method executed by a controller in a medical system which comprises a memory which stores data relating to medical processes including at least a placement process, an imaging process, and a surgery process, and a robotic operation table including a table top on which a patient is to be placed and a robot arm supporting the table top. The method may include: positioning the table top, by the robot arm, at a placement position where the patient is placed on the table top, by reading the data relating to the placement process from the memory and giving a command to the robot arm; positioning the table top, by the robot arm, at a surgery position where surgery is performed on the patient, by reading the data relating to the surgery process from the memory and giving a command to the robot arm; and positioning the table top, by the robot arm, at an imaging position where an image of the patient is taken by a medical imaging device, by reading the data relating to the imaging process from the memory and giving a command to the robot arm. The robot arm may include a plurality of movable elements, and a plurality of joints connecting the plurality of movable elements, a plurality of electric actuators provided corresponding to the plurality of joints respectively, and a plurality of position detectors provided corresponding to the plurality of joints respectively, wherein at least one of the plurality of joints is a horizontally-rotating joint which couples two movable elements among the plurality of movable elements to each other so as to be rotatable about a vertical axis.

A robotic operation table according to one or more embodiments may include a table top on which a patient is to be placed and a robot arm supporting the table top. The robot arm includes a plurality of movable elements, a plurality of joints connecting the plurality of movable elements, a plurality of electric actuators provided corresponding to the plurality of joints respectively, and a plurality of position detectors provided corresponding to the plurality of joints respectively, wherein at least one of the plurality of joints is a horizontally-rotating joint which couples two movable elements among the plurality of movable elements to each other so as to be rotatable about a vertical axis. The robot arm is configured, based on data relating to medical processes including at least a placement process, an imaging process, and a surgery process stored in a memory, to position the table top to positions corresponding to the medical processes including a placement position where the patient is placed on the table top, an imaging position where an image of the patient is taken by a medical imaging device, and a surgery position where surgery is performed on the patient.

### BRIEF DESCRIPTION OF THE DRAWINGS

FIG. 1 is a diagram illustrating a perspective view of a robotic table according to a first example configuration.

FIG. 2 is a diagram illustrating a side view of the robotic table according to the first example configuration.

FIG. 3 is a diagram illustrating a conceptual view of an actuator, a position detector, and a braking mechanism configured as a single unit.

FIG. 4 is a diagram illustrating a perspective view of a robotic table according to a variation of the first example configuration.

FIG. 5 is a diagram illustrating a side view of a robotic table configured to have a minimum degree of freedom according to the first example configuration.

FIG. 6 is a diagram illustrating a plan view of a medical room where the robotic table according to the first example configuration is placed, and shows a state in which the table top is located at a first position.

FIG. 7 is a diagram illustrating a plan view of the medical room where the robotic table according to the first example configuration of a robot arm is placed, and shows the table top in the middle of being transferred from the first position to a second position.

FIG. 8 is a diagram illustrating a plan view of the medical room where the robotic table according to the first example configuration of the robot arm is placed, and shows a state in which the table top is located at the second position.

FIG. 9 is a diagram illustrating a side view of a robotic table according to a second example configuration.

FIG. 10 is a diagram illustrating a side view of the robotic table according to the second example configuration, in which the robotic table is moved upward.

FIG. 11 is a diagram illustrating a side view of a robotic table according to a variation of the second example configuration.

FIG. 12 is a diagram illustrating a side view of the robotic table according to the variation of the second example configuration, and shows the transition state of the robotic table moved up and down.

FIG. 13 is a diagram illustrating a plan view of a medical room where the robotic table according to the second example configuration is placed, and shows a state in which the table top is located at a first position.

FIG. 14 is a diagram illustrating a plan view of the medical room where the robotic table according to the second example configuration is placed, and shows the table top in the middle of being transferred from the first position to a second position.

FIG. 15 is a diagram illustrating a plan view of the medical room where the robotic table according to the second example configuration is placed, and shows a state in which the table top is located at the second position.

FIG. 16A is a diagram illustrating a side view of an example slide mechanism used for a robotic table according to a third example configuration, in which the robotic table has a lower surface provided with a groove in which the slide mechanism is fitted.

FIG. 16B is a diagram illustrating a plan view of the example slide mechanism used for the robotic table according to the third example configuration, in which both sides of the groove are provided with racks each having a plurality of teeth.

FIG. 17 is a diagram illustrating a side view of the robotic table according to the third example configuration.

FIG. 18 is a diagram illustrating a perspective view of an MRI apparatus.

FIG. 19 is a diagram illustrating a perspective view of the case in which the robotic table according to the third example configuration is employed in intraoperative MRI, and shows a state in which the table top is located at a surgery position.

FIG. 20 is a diagram illustrating a perspective view of the case in which the robotic table according to the third example configuration is employed in intraoperative MRI, and shows a state in which the table top is located at an imaging preparation position.

FIG. 21 is a diagram illustrating a perspective view of the case in which the robotic table according to the third example configuration is employed in intraoperative MRI, and shows a state in which the table top is located at an imaging position.

FIG. 22 is a diagram illustrating a plan view of the medical room where the robotic table according to the first example configuration is placed, and shows a state in which the table top is located at a third position.

FIG. 23 is a diagram illustrating a plan view of the medical room where the robotic table according to the second example configuration is placed, and shows a state in which the table top is located at the third position.

FIG. 24A is a diagram illustrating a perspective view of the robotic table according to the second example configuration provided with a slide mechanism, and shows a state in which the table top is located at a surgery position as a first position.

FIG. 24B is a diagram corresponding to FIG. 24A and illustrating a plan view of the robotic table according to the second example configuration provided with the slide mechanism, and shows the state in which the table top is located at the surgery position as the first position.

FIG. 24C is a diagram illustrating a perspective view of the robotic table according to the second example configuration provided with the slide mechanism, and shows a state in which the table top arrives at an imaging preparation position.

FIG. 24D is a diagram corresponding to FIG. 24C and illustrating a plan view of the robotic table according to the second example configuration provided with the slide mechanism, and shows the state in which the table top arrives at the imaging preparation position.

FIG. 24E is a diagram illustrating a perspective view of the robotic table according to the second example configuration provided with the slide mechanism, and shows a state in which the table top arrives at an imaging position (a second position).

FIG. 24F is a diagram corresponding to FIG. 24E and illustrating a plan view of the robotic table according to the second example configuration provided with the slide mechanism, and shows the state in which the table top arrives at the imaging position (the second position).

FIG. 25A is a diagram illustrating a plan view of a robotic table configured to allow the robot arm to protrude from under the table top while the robotic table is taking a position requiring a minimum space in a medical room, for showing dimensions of the robotic table.

FIG. 25B is a diagram illustrating a plan view of a robotic table configured such that the robot arm is completely hidden under the table top while the robotic table is taking a space-saving position, for showing dimensions of the robotic table.

FIG. 26 is a block diagram illustrating a configuration of a medical process management system.

FIG. 27 is a diagram generally illustrating a monitor displaying an example series of medical processes.

FIG. 28 is a diagram illustrating an appearance of an operating device.

FIG. 29 is a diagram generally illustrating a monitor displaying another example series of medical processes.

### DESCRIPTION OF EMBODIMENTS

One or more embodiments disclosed herein relate to system construction and process control for treatments (e.g., radiotherapy, a catheter treatment, and a hybrid operation) and inspection (e.g., medical imaging) which are carried out by moving a target placed on a table top (e.g., a robotic table) supported by a robot arm. Medical care is a concept including treatment and inspection. For example, if a robotic table is used for the radiotherapy or the catheter treatment, the robotic table is a robotic treatment table. If a robotic table is used for the hybrid operation, the robotic table is a robotic operation table. Thus, the term “treatment” used herein includes surgery, such as radiotherapy, a catheter treatment, and a tumor removal surgery. Likewise, the terms “robotic treatment table” and “treatment room” are concepts which also include a robotic operation table and an operating room, respectively. The term “medical room” is a concept which also includes a treatment room, an operating room, an inspection room, and so on.

[Configuration of Robotic Table]

### First Example Configuration

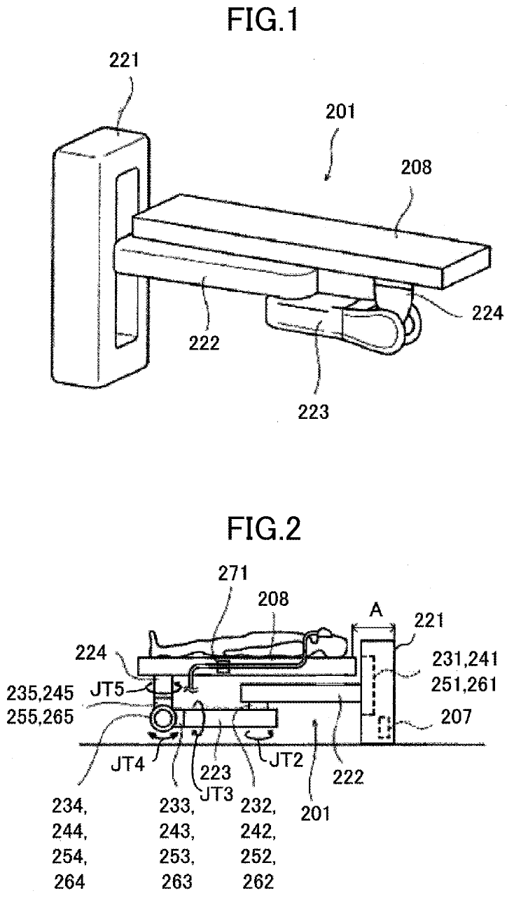

FIG. 1 is a diagram illustrating a perspective view of a robotic table according to a first example configuration, and FIG. 2 is a diagram illustrating a side view of the robotic table. A robot arm 201 used for the robotic table has multiple degrees of freedom (i.e., three or more degrees of freedom), and has a distal end supporting a table top 208 on which a target is placed. The table top 208 and the robot arm 201 form the robotic table.

As illustrated in FIG. 2, the robot arm 201 includes a base 221, a plurality of movable elements (first to third movable elements 222 to 224 in the present example configuration), and a plurality of joints (first to fifth joints 231 to 235 in the present example configuration).

The base 221 and one end portion of the first movable element 222 are coupled together by the first joint 231 traveling vertically straight, which enables the first movable element 222 to move in a first axial direction (i.e., in a vertical direction). The other end portion of the first movable element 222 and one end portion of the second movable element 223 are coupled together by a horizontally-rotating joint, which enables the second movable element 223 to rotate about a second axis (in the vertical direction). The third to fifth joints 233 to 235 between the second movable element 223 and the third movable element 224 are rotating joints which rotate about third to fifth axes, respectively. The third axis corresponds to a direction in which the second movable element 223 extends. The fourth axis corresponds to a direction orthogonal to the third axis about which the third joint 233 rotates. The fifth axis corresponds to a direction orthogonal to the fourth axis about which the fourth joint 234 rotates.

Each of the first movable element 222 and the second movable element 223 is a rod-like member extending in a particular direction, with its length appropriately designed according to a required range of movement of the robot arm 201. The “one end portion” of a movable element extending in a particular direction refers to either one of the two end regions when the movable element is equally divided into three regions in the particular direction (i.e., the longitudinal direction). The “other end portion” of the movable element extending in the particular direction refers to the end portion opposite to the one end portion of the two end regions of the three equally-divided regions of the movable element in the particular direction (i.e., the longitudinal direction). If it is simply called the “end portion,” it refers to either the one end portion or the other end portion. The portion between both of the two end portions is called a “middle portion.”

The first movable element 222 moves up and down, while staying parallel to the horizontal plane. The second movable element 223 rotates about the second axis, while staying parallel to the first movable element 222. This configuration does not require the second actuator 242 to compensate for the gravity in the vertical direction, and the motor may thus be reduced in size. This configuration is advantageous in downsizing the robot arm 201, and is advantageous in introducing the robot arm 201 in the medical settings where only a limited space is available, or in giving a larger space for treatment and surgery. For example, a ball screw may be employed as a configuration of the first joint 231 to which load is applied.

Further, the robotic table according to the present example configuration is configured such that the table top 208 does not come in contact with the robot arm 201, no matter how much (e.g., by 360 degrees) the table top 208 is rotated while keeping the table top 208 parallel to the horizontal plane, in a state in which particular directions (i.e., the longitudinal directions) of the first movable element 222 and the second movable element 223, which are coupled together at their end portions by a horizontally-rotating joint, are parallel to each other when viewed from vertically above. Specifically, the robotic table according to the present example configuration is configured such that, in a state in which the first movable element 222 and the second movable element 223, which are coupled together at their end portions by a horizontally-rotating joint, and the table top 208 are arranged parallel to the horizontal plane, the table top 208 is not level with the other movable elements and is located at the top. In other words, in a state in which the distal end of the robot arm 201 is located at the lowermost position of its motion range and the table top 208 takes a position parallel to the horizontal plane, the first and second movable elements of the robot arm 201 are lower than the lower surface of the table top 208. Further, in the present example configuration, the base 221 is higher than the lower surface of the table top 208 in order to provide a greater range of adjustment for the vertical movement of the table top 208, even in a state in which the distal end of the robot arm 201 is located at the lowermost position of its motion range and the table top 208 takes a position parallel to the horizontal plane. These configurations allow the movable elements of the robot arm 201 to be located and housed under the table top 208, and hence allow effective use of a limited space in the medical settings while ensuring a broad range for the vertical movement of the table top 208.

Further, for the purpose of space saving and in consideration of the size of the robot arm 201 enough to maintain the strength for supporting the table top 208, the dimension A (see FIG. 2) in the longitudinal direction of the table top 208 where the robot arm 201 is not hidden under the table top 208 is preferably one fourth (i.e., ¼) or less of the longitudinal dimension of the table top 208.

Advantages of this configuration can be clearly seen from FIGS. 6 to 8 illustrating the movement of the robotic table according to the first example configuration. As illustrated in FIG. 6, the robotic table according to the present example configuration can take a position in which the respective movable elements and the table top 208 overlap one another when viewed from vertically above. Thus, even in the case, for example, where the table top is located as close to the base as possible in order to keep space for treatment, the movable elements do not constitute obstacles.

Preferably, the width of the table top 208 is greater than the width of each of the movable elements of the robot arm 201. For example, it is preferable that in a state in which particular directions (i.e., the longitudinal directions) of the first movable element 222 and the second movable element 223, which are coupled together at their end portions by a horizontally-rotating joint, and a particular direction (i.e., the longitudinal direction) of the table top 208 are parallel to one another when viewed from vertically above, the first movable element 222 and the second movable element 223 be hidden under the table top 208 in the direction (i.e., the width direction of the table top 208) orthogonal to the particular direction (i.e., the direction in which the longitudinal directions of the first movable element 222, the second movable element 223, and the table top 208 are parallel to each other) at portions where the table top 208 overlaps with the first movable element 222 and the second movable element 223 in the particular direction (i.e., the longitudinal direction) when viewed from vertically above. In this configuration, portions of the robot arm 201 (that is, in the example of FIG. 2, all of the first movable element 222 other than the one end portion thereof, and all of the second movable element 223 and the third movable element 224) which overlap with one another in the longitudinal direction of the table top 208 are housed under the table 208 at least in the width direction of the table top 208 (i.e., the direction orthogonal to the particular direction in which the table top 208 extends) (see, e.g., FIG. 6).

In the examples illustrated in FIGS. 1 and 2, one (i.e., the first movable element 222) of the two movable elements (namely, the first movable element 222 and the second movable element 223) which are coupled together at their end portions by a horizontally-rotating joint is directly coupled to the base 221. However, the movable element may also be indirectly coupled to the base via another horizontally-rotating joint or a vertically-rotating joint. In this case, as well, the advantages of ensuring a larger space and downsizing the robot arm can be achieved, as long as the above-described positional relationship is maintained and the plurality of movable elements are housed under the table top 208.

The third movable element 224 is provided at the distal end of the robot arm 201. In the present example configuration, the distal end of the robot arm 201 is fixed on a lower surface of the one end portion of the table top 208 extending in the particular direction. This configuration allows the robot arm 201 to move such that the other end of the table top 208 is positioned as far away from the base 221 as possible. Supporting the table top 208 at its one end portion increases the movable range of the table top 208. However, the table top 208 may be supported at its middle portion if a priority is placed on the supporting strength.

The robot arm 201 includes: a plurality of actuators (first to fifth actuators 241 to 245 in the present example configuration) associated with the first to fifth joints 231 to 235 to move or rotate the first to third movable elements 222 to 224; a plurality of position detectors (first to fifth position detectors 251 to 255 in the present example configuration) built in the respective joints to detect the positions of the respective movable elements; and a robot arm controller 207 (see FIG. 2) which controls the actuation of the respective actuators. The robot arm controller 207 is provided in the base 221, but may also be an independent external device, for example.

The first to fifth actuators 241 to 245 are servomotors, for example. Encoders, resolvers, and potentiometers may be used as the position detectors.

Preferably, the robot arm 201 further includes first to fifth electromagnetic brakes 261 to 265 associated with the first to fifth joints 231 to 235. If the robot arm 201 does not include any electromagnetic brakes, the posture of the robot arm 201 is maintained by actuating the plurality of actuators 241 to 245. If the robot arm 201 includes the electromagnetic brakes, the posture of the robot arm 201 may be maintained by turning the electromagnetic brakes on even if some of the actuators are turned off.

In the case where the electromagnetic brakes are provided, each of the first to fifth electromagnetic brakes 261 to 265 is configured to turn its brake function on when no drive current is supplied to the associated one of the actuators, and to turn its brake function off when a drive current is supplied to the actuator.

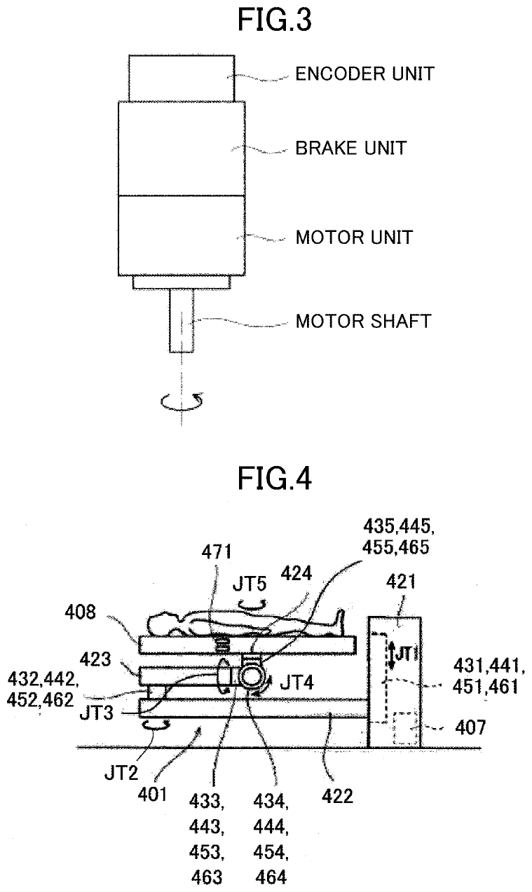

In many cases, a motor functioning as the actuator, an encoder functioning as the position detector, and the brake are integrated together as a unit as illustrated in FIG. 3. Further, each of the first to fifth actuators 241 to 245 is provided with a deceleration mechanism, a coupling, etc., for power transmission.

In the example illustrated in FIG. 2, the first movable element 222 is coupled by the horizontally-rotating joint 232 so as to be located above the second movable element 223.

Illustrated in FIG. 4 as a variation of the present example configuration is a robot arm 401, the first movable element 422 of which is coupled by a horizontally-rotating joint 432 so as to be located below the second movable element 423.

In this variation, the base 421 and one end portion of the first movable element 422 are coupled together by the first joint 431 traveling vertically straight, which enables the first movable element 422 to move in a first axial direction (i.e., in a vertical direction). The other end portion of the first movable element 422 and one end portion of the second movable element 423 are coupled together by a horizontally-rotating joint, which enables the second movable element 423 to rotate about a second axis (in the vertical direction) above the first movable element 422. Third to fifth joints 433 to 435 between the second movable element 423 and the third movable element 424 are rotating joints which rotate about third to fifth axes, respectively. The third axis corresponds to a direction in which the second movable element 423 extends. The fourth axis corresponds to a direction orthogonal to the third axis about which the third joint 433 rotates. The fifth axis corresponds to a direction orthogonal to the fourth axis about which the fourth joint 434 rotates.

The third movable element 424 is provided at the distal end of the robot arm 401. In the present example configuration, the distal end of the robot arm 401 is fixed on a lower surface of a middle portion of the table top 408 extending in the particular direction. This configuration allows supporting the table top 408, while placing a priority on the supporting strength. Of course, the table top 408 may be supported at its one end portion to place a priority on the movable range of the table top 408. In that case, however, it is necessary to determine the dimensions of the respective movable elements 422 to 424 and the table top 408 appropriately in order to avoid contact with the robot arm 401 even when the table top 408 is freely rotated while staying parallel to the horizontal plane.

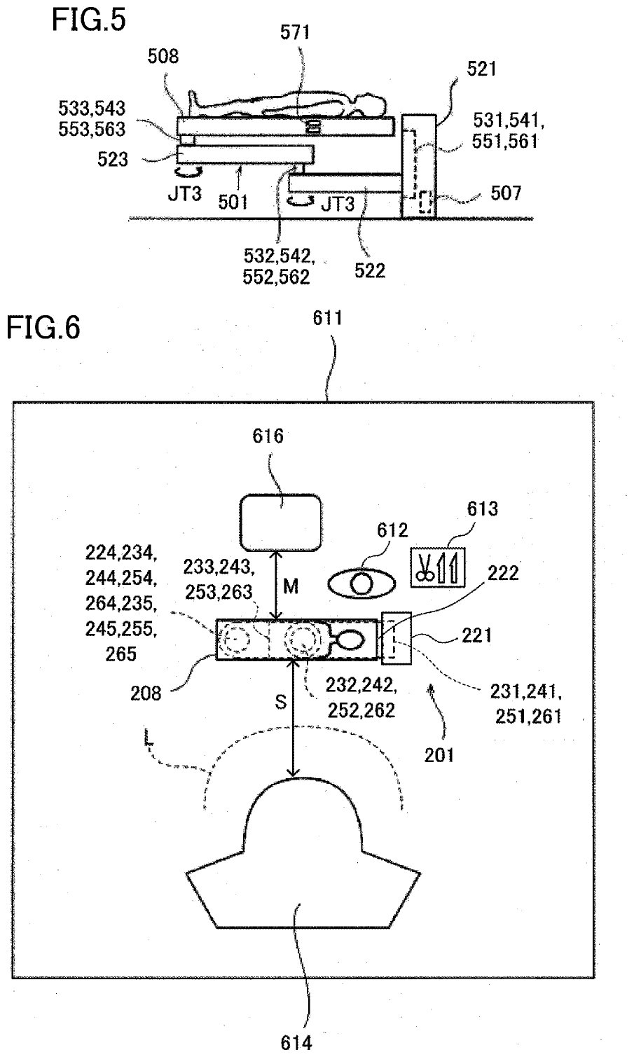

The robot arms 201 and 401 illustrated in FIGS. 2 and 4 have five degrees of freedom. However, the degrees of freedom of the robot arm of one or more embodiments disclosed herein do not have to be five, and may be four or less or six or more. Nevertheless, it is preferable that the degrees of freedom of the robot arm be three or more so that the table top 208 and 408 can move at least in a straight manner in the room. FIG. 5 illustrates an example robotic table having three degrees of freedom. In FIG. 5, the robot arm 501 is comprised of a base 521 and two movable elements 522 and 523. The base 521 and one end portion of the first movable element 522 are coupled together by a first joint 531 traveling vertically straight, which enables the movable element 522 to move in a first axial direction (i.e., in a vertical direction). The other end portion of the first movable element 522 and one end portion of the second movable element 523 are coupled together by a second joint 523, which is a horizontally-rotating joint enabling the second movable element 523 to rotate about a second axis (i.e., in the vertical direction). The other end of the second movable element 523 serves as the distal end of the robot arm 501, and is coupled to one end portion of the table top 508 by a third joint 533, which is a horizontally-rotating joint.

The robotic table having the above configuration makes it possible to move the table top 208, 408, and 508, on which a target has been placed, to a target position, such as an inspection position and a treatment position, accurately and quickly, thus achieving significant improvement in the efficiency of the inspection and treatment in the medical settings. For example, compared to the configuration in which a table top with casters is used to move the patient, the table top 208, 408, and 508 can be moved more smoothly without shaking the patient too much, and may be prevented from being tangled with a lot of cords of medical equipment and the tubes of medical instruments which run on the floor of the medical room, and may be prevented from being wobbled by stepping over the cords and tubes. Thus, safety and transfer efficiency can be improved.

Further, in the robotic table according to the present example configuration, the movable elements indicated by the reference characters 223, 423, and 523 are coupled to the table top indicated by the reference character 508 by the joints indicated by the reference characters 232, 432, 532, and 533, each of which is a horizontally-rotating joint that enables the movable elements and the table top to rotate while always staying parallel to the horizontal plane. This configuration thus provides greater stiffness, compared to the case where each of the movable elements and the table top are coupled by a vertically-rotating joint. Specifically, if the movable element and the table top are coupled together by a vertically-rotating joint, the posture may not be completely maintained by only the control by the actuator, and warpage may occur, due to, for example, the weight of the placed target, while the table top is being moved or staying in a certain posture. The horizontally-rotating joint, on the other hand, does not rotate in the vertical direction, and therefore such warpage hardly occurs. Moreover, it is not necessary to take a vertical rotation into account at a portion where the horizontally-rotating joint, which always enables rotation parallel to the horizontal plane, is provided. Thus, the electromagnetic brake may be omitted even in consideration of a situation in which the power is turned off. In this manner, the present example configuration has greater stiffness and also contributes to providing a larger treatment space, and is designed to be more suitable as a robotic table used in a medical room.

Examples of the target positions of the robotic table include: a placement position where a target, such as a human being and an animal, is placed on the robotic table; an inspection position where an inspection is conducted using specific inspection equipment or measurement equipment; an imaging position where an image of a specific site of the placed target is taken by CT, MRI, angiography, etc.; a treatment preparation position where a nurse or other staff gives medical attention to the patient before treatment; and a treatment position (including the surgery position) where a doctor and an assistant give treatment. The robotic table may be moved to different positions even for the same purpose, if, for example, different treatments need to be given at a plurality of sites. Specifically, the robotic table may be used, for example, as follows: the table top may be moved to the inspection position to inspect the placed target for any objects, like an implant, which affect MRI, before being moved to the MRI scanning position; the table top may be moved to the inspection position to detect an amount of radioactive substances deposited using a detector, before the patient, who is a placed target, is moved to the surgery position; the patient, who is a placed target, may be moved to the inspection position to check his/her skin condition, before the patient is moved to the surgery position for skin surgery; and the table top may be moved to the imaging position for brain tomography by an MRI apparatus, before being moved to the surgery position for surgery removing a brain tumor.

The movements of the table top 208 supported by the robot arm 201 of the present example configuration between the plurality of positions will be described with reference to FIGS. 6 to 8.

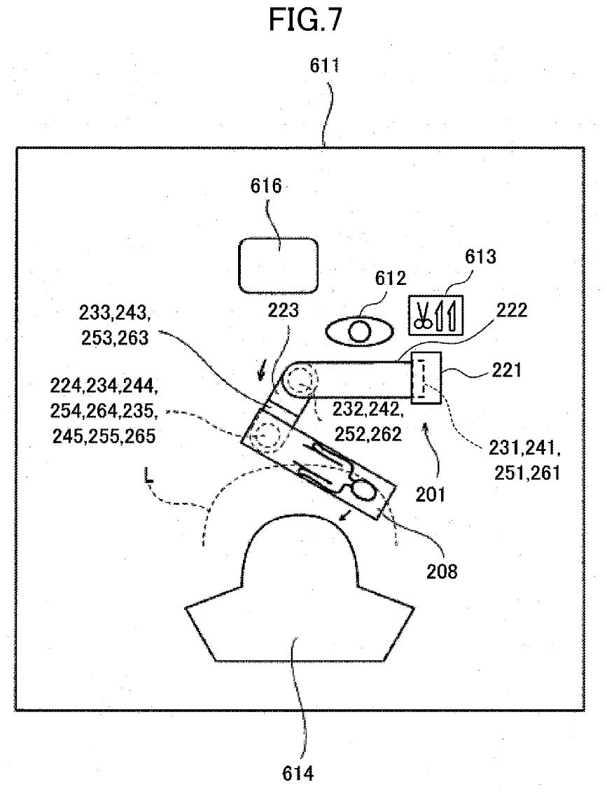

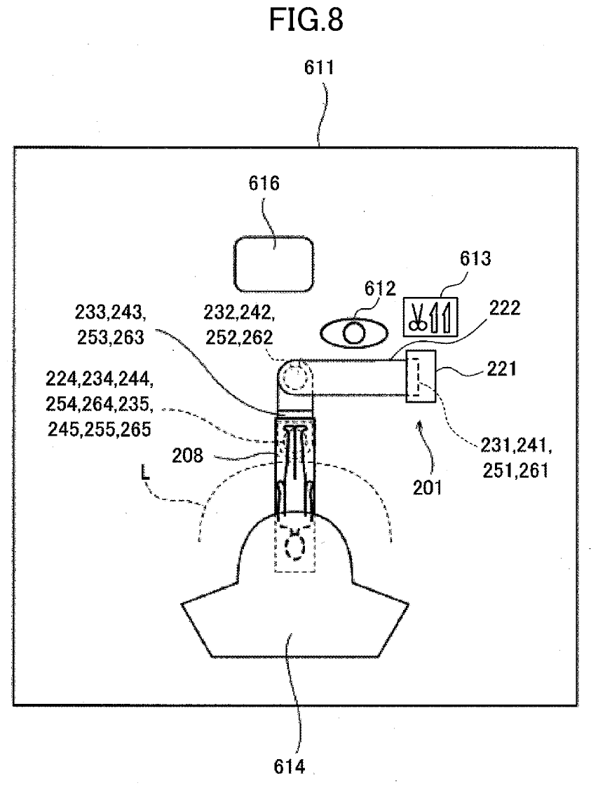

FIG. 6 illustrates a state in which the table top 208 is located at the placement position (i.e., a first position) in the process of moving a subject, who is a placed target, from the placement position to the inspection position. FIG. 7 illustrates a state in which the second movable element 223 and the table top 208 are moved by the control of the robot arm controller 207 as the arrows indicate (in some cases, the first movable element 222, too, is moved in the vertical direction to have its height adjusted, and the table top 208 is rotated about the third axis and/or the fourth axis to have its tilt with respect to the longitudinal direction and/or the width direction of the table top finely adjusted), causing the head of the subject to move toward the inspection device 614 from an oblique angle. FIG. 8 illustrates a state in which the table top 208 is inserted in the inspection device 614, and the subject has arrived at the inspection position (i.e., a second position). Note that the position (i.e., the first position) of the table top 208 illustrated in FIG. 6 can also be the treatment position. From the inspection position (i.e., the second position) illustrated in FIG. 8, the respective movable elements move in reverse direction until the table top 208 returns to the position illustrated in FIG. 6, where a doctor 612 can give a treatment based on the result of the inspection that has just been conducted.

The robot arm 501 illustrated in FIG. 5, as well, enables the table top 508 to follow a similar path. Turning to the robot arm 401 illustrated in FIG. 4, the table top 408 can arrive at the inspection position by rotating the second movable element 423 and the table top 408 in the direction opposite to the direction indicated by the arrows shown in FIG. 7 (in some cases, the first movable element 422, too, moves in the vertical direction to have its height adjusted, and the table top 408 is rotated about the third axis and/or the fourth axis to have its tilt with respect to the longitudinal direction and/or the width direction of the table top finely adjusted).

The movement of the table top 208, 408, and 508 by the robot arm 201, 401, and 501 between the respective positions may be achieved by, for example, giving an instruction to move the movable elements of the robot arm 201, 401, and 501 to the robot arm controller 207, 407, and 507 through an instruction device (an operating device), such as a teaching pendant. Alternatively, the respective positions, such as the treatment position and the inspection position, may be stored in the robot arm controller 207, 407, and 507 in advance. In this configuration, the movable elements work in such a manner that allows the table top to move to the target position in the shortest time by simply giving, for example, a forward-movement instruction to the robot arm controller or while the robot arm controller is receiving the forward-movement instruction. The table top 208, 408, and 508 can thus be moved to the target position more quickly and smoothly. Further, the target position and some points on the intended path to the target position may be designated. In this configuration, the table top may automatically travel along the intended path and arrive at the target position by simply giving, for example, a movement start instruction or keep giving a movement continuation instruction to the robot arm controller 207, 407, and 507. To record the respective positions, the respective positions may be directly stored by actually guiding the robot arm 201, 401, and 501 to the target position through the teaching pendant. Alternatively, the respective positions may be designated by inputting their x, y and z coordinates. Note that the instruction device is not limited to the teaching pendant, and may also be a hand-held, a remote controller, or the like.

### Second Example Configuration

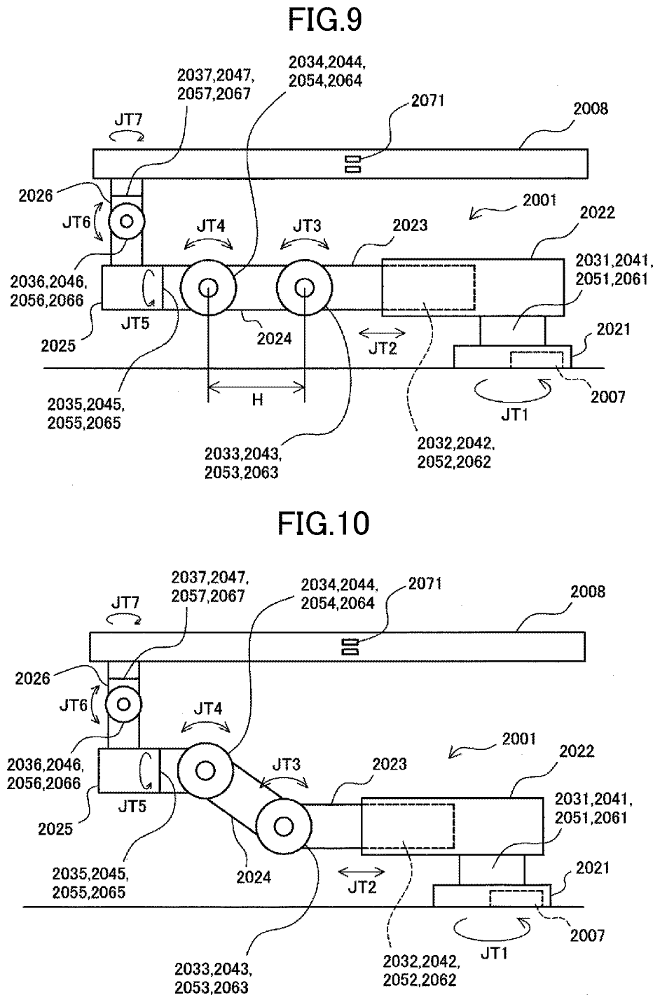

FIG. 9 is a diagram illustrating a side view of a robotic table according to a second example configuration. A robot arm 2001 used for the robotic table has multiple degrees of freedom (i.e., three or more degrees of freedom), and has a distal end supporting a table top 2008 on which a target is placed. The table top 2008 and the robot arm 2001 form the robotic table.

As illustrated in FIG. 9, the robot arm 2001 includes a base 2021, a plurality of movable elements (first to fifth movable elements 2022 to 2026 in the present example configuration), and a plurality of joints (first to seventh joints 2031 to 2037 in the present example configuration).

The base 2021 and one end portion of the first movable element 2022 are coupled together by a first joint 2031, i.e., a horizontally-rotating joint, which enables the first movable element 2022 to move in a first axial direction (i.e., in a vertical direction). The other end portion of the first movable element 2022 has an opening at least on the other end portion side in a particular direction. One end of the second movable element 2023 is fitted in the opening. The first movable element 2022 and the second movable element 2023 are coupled together by a joint traveling straight. This configuration allows the second movable element 2023 to move in a second axis direction (i.e., the horizontal direction). The other end portion of the second movable element 2023 and one end portion of the third movable element 2024 are coupled together by a vertically-rotating joint, which enables the third movable element 2024 to rotate about a third axis orthogonal to both of the longitudinal direction (i.e., the extending direction of the third movable element 2024) and the vertical direction. The other end portion of the third movable element 2024 and one end portion of the fourth movable element 2025 are coupled together by a vertically-rotating joint, which enables the fourth movable element 2025 to rotate about a fourth axis orthogonal to both of the longitudinal direction (i.e., the extending direction of the fourth movable element 2025) and the vertical direction and parallel to the third axis. The rotation of the third axis and the rotation of the fourth axis can be controlled independently. For example, the third and fourth movable elements 2024 and 2025 may be configured such that when the third movable element 2024 makes a 15 degree clockwise rotation about the third axis, the fourth movable element 2025 makes a 15 degree counterclockwise rotation about the fourth axis in synchronization (see FIG. 10). As a result, the fourth movable element 2025 is movable up and down, with the entire fourth movable element 2025 staying parallel to the horizontal plane. The fifth to seventh joints 2035 to 2037 between the fourth movable element 2025 and the fifth movable element 2026 are rotating joints which rotate about fifth to seventh axes, respectively. The fifth axis corresponds to a direction in which the fourth movable element 2024 extends. The sixth axis corresponds to a direction orthogonal to the fifth axis about which the fifth joint 2035 rotates. The seventh axis corresponds to a direction orthogonal to the sixth axis about which the sixth joint 2036 rotates.

Each of the first to fourth movable elements 2022 to 2025 is a rod-like member extending in the particular direction, with its length appropriately designed according to a required range of movement of the robot arm 2001 and a range of movement of the table top 2008 in the vertical direction. In the present example configuration, the up and down movement of the table top 2008 in the vertical direction is realized by two rotating joints (i.e., the third vertically-rotating joint 2033 and the fourth vertically-rotating joint 2034) capable of being positioned on the same horizontal plane (i.e., at the same height). Thus, the base does not need to be as high as the base in the first example configuration. That is, the traveling range of the table top 2008 in the vertical direction is adjustable not based on the height of the base in the vertical direction, but based on the length of the third movable element 2024. In this manner, the two movable elements (2023 and 2024, or 2024 and 2025), coupled together by the vertically-rotating joint (2033 and 2034) to move the table top 2008 in the vertical direction, are located on the same horizontal plane, while taking a particular position (e.g., while the robot arm 2001 takes a position where the table top 2008 is located at the lowermost position in the range of movement in the vertical direction). Thus, the table top can be further lowered in height, making it possible to ensure treatment at a lower position and placement of a target at a lower position. The configuration of the base 2021 capable of hiding under the table top 2008 is advantageous in introducing the robot arm in the medical settings where only a limited space is available, or in ensuring a larger space for treatment and surgery. The range of adjustment in the height of the table top 2008 depends on the length H of the third movable element. The height H is therefore determined in consideration of the range of movement of the table top in the vertical direction.

The two movable elements do not have to be coupled together by the vertically-rotating joint at their end portions as illustrated in FIG. 9. For example, the two movable elements may be coupled together at their side surfaces by the vertically-rotating joint. The configuration in which the movable elements coupled together by a vertically-rotating joint are located on the same horizontal plane does not necessarily require the linear motion joint to be used together. For example, such a configuration may also be used as a substitute for the vertically traveling joint used in the first and second example configurations, and is not limited to the case described in the present example configuration. The configuration is an independent feature for achieving a space saving robotic table.

In the first example configuration, the robot arm is configured such that the movable elements are coupled together at their end portions by a horizontally-rotating joint. Thus, the movable elements overlap each other in the vertical direction. On the other hand, a joint traveling horizontally straight is employed in the robot arm of the present example configuration. Thus, the overlapping does not occur, which is more advantageous in placing the table top 2008 at a lower position.

Further, the robotic table of the present example configuration is configured to prevent the table top 2008 from coming into contact with the robot arm 2001, even when the table top 2008 is moved up and down (i.e., in the vertical direction) with the table top 2008 maintained parallel to the horizontal plane, or no matter how much (e.g., 360 degrees) the table top 2008 is rotated. Thus, in the present example configuration, the table top and the robot arm do not come into contact with each other, no matter what posture the robot arm has, or how much the table top 2008 is rotated, as long as the table top 2008 is maintained parallel to the horizontal plane.

It is preferable that the width of the table top 2008 be greater than the width of each of the movable elements of the robot arm 2001 and the width of the base, and therefore that the entire robot arm 2001, including the base 2021, be hidden under the table top 2008 when viewed from vertically above. For example, it is preferable that all the movable elements and the base 2021 may be hidden under the table top 2008 when the table top 2008 is viewed from vertically above, in a state in which the longitudinal direction of the table top 2008 and the particular directions of the first and second movable elements 2022 and 2023 are parallel to each other when viewed from vertically above.

In the present example configuration, the fifth movable element 2026 is provided at the distal end of the robot arm 2001. In FIGS. 9 and 10, the distal end of the robot arm 2001 is fixed on a lower surface of an end of the table top 2008 extending in the particular direction. The range of movement of the table top 2008 can thus be increased.

The definitions of the “one end portion,” “other end portion,” “end portion” and “middle portion” as adopted in the above description are the same as, or similar to, those adopted in the first example configuration.

The robot arm 2001 includes: a plurality of actuators (first to seventh actuators 2041 to 2047 in the present example configuration) associated with the first to seventh joints 2031 to 2037 to move or rotate the first to fifth movable elements 2022 to 2026; a plurality of position detectors (first to seventh position detectors 2051 to 2057 in the present example configuration) built in the respective joints to detect the positions of the respective movable elements; and a robot arm controller 2007 (see FIG. 9) which controls the actuation of the respective actuators. In the present example configuration, the robot arm controller 2007 is provided in the base 2021, but may also be an independent external device, for example.

The first to seventh actuators 2041 to 2047 are servomotors, for example. Similarly to the first to third example configurations, encoders, resolvers or potentiometers may be used as the position detectors.

Preferably, the robot arm 2001 further includes first to seventh electromagnetic brakes 2061 to 2067 associated with the first to seventh joints 2031 to 2037. If the robot arm 2001 does not include any electromagnetic brakes, the posture of the robot arm 2001 is maintained by actuating the plurality of actuators 2041 to 2047. If the robot arm 2001 includes the electromagnetic brakes, the posture of the robot arm 2001 may be maintained by turning the electromagnetic brakes on even if some of the actuators are turned off.

In the case where the electromagnetic brakes are provided, each of the first to seventh electromagnetic brakes 2061 to 2067 is configured to turn its brake function on when no drive current is supplied to the associated one of the actuators, and to turn its brake function off when a drive current is supplied to the actuator.

Similarly to the first example configuration, in many cases, a motor functioning as the actuator, an encoder functioning as the position detector, and the brake are integrated together as a unit as illustrated in FIG. 3. Further, each of the first to seventh actuators 2041 to 2047 is provided with a deceleration mechanism, a coupling, etc., for power transmission.

(Variations)

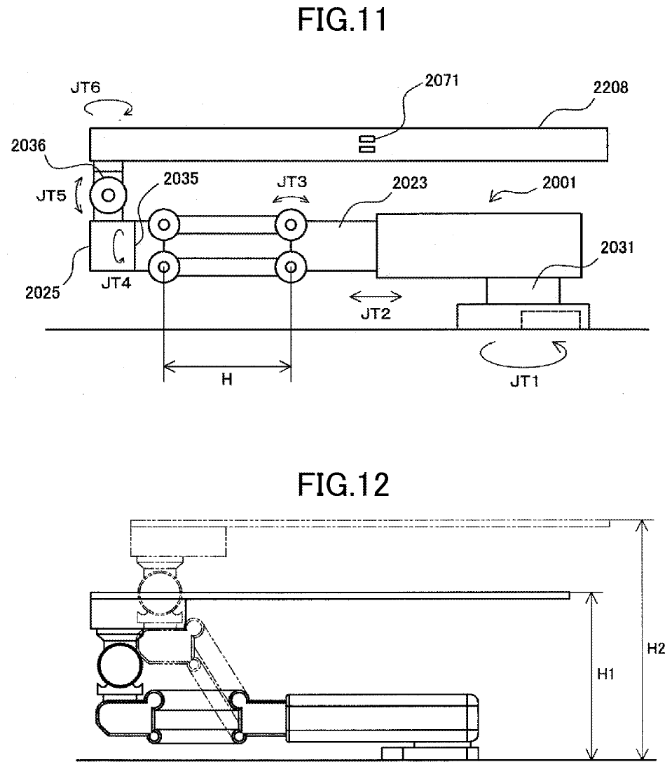

FIG. 11 is a diagram illustrating a si de view of a variation according to the second example configuration. The present variation differs from the second example configuration in that the third movable element 2024 and the third and fourth joints are replaced with a parallel link mechanism. That is, the third movable element 2024 is replaced with a movable element comprised of two links (i.e., upper and lower links). One end portion of this movable element is coupled to the second movable element 2023 through an axis parallel to the third axis. The other end portion of this movable element is coupled to the fourth movable element 2025 through an axis parallel to the fourth axis.

The parallel link is provided with an actuator associated with only one of the four rotational shafts which consists of two rotational shafts connected to the second movable element 2023 and two rotational shafts connected to the fourth movable element 2025. In the present variation illustrated in FIG. 11, the actuator (and a position detector and a brake) is provided at the upper rotational shaft of the rotation shafts which are connected to the second movable element 2023.

The parallel link of the present example configuration serves as an interacting mechanism in which a clockwise rotation of the rotational shaft provided with the actuator causes the other rotational shaft on the same side to rotate clockwise by the same rotational amount, and causes the two rotational shafts on the opposite side to rotate counterclockwise by the same rotational amount. As a result, the fourth movable element 2025 is movable up and down in the vertical direction, while keeping the same state with respect to the horizontal plane. FIG. 12 is a diagram illustrating a side view of the table top 2008 moved up and down in the present variation.

With the parallel link mechanism employed in the present variation, it is not the rotational shifts provided at the second movable element 2023 of the parallel link, but the rotational shafts provided at the fourth movable element 2025, that receive the weight of the target placed on the table top 2008, while the table top 2008 moves up and down in the vertical direction. It is therefore possible to reduce torque for moving the table top 2008 up and down in the vertical direction. The actuator for driving the parallel link can thus be reduced in size, and hence the robot arm 2001 can be reduced in size. Reduction in size of the robot arm 2001 is advantageous in the configuration in which the entire robot arm 2001 is housed under the table top 2008.

Note that one joint can be omitted from the present variation since the number of actuators (and the position detector and the brake) is decreased by one. That is, the fourth joint 2034 in FIG. 9 is omitted, and the fifth to seventh joints in FIG. 9 function as the fourth to sixth joints in FIG. 10.

The robotic table having the above configuration makes it possible to move the table top 2008, on which a target has been placed, to a target position, such as an inspection position and a treatment position, accurately and quickly, thus achieving significant improvement in the efficiency of the inspection and treatment in the medical settings. For example, compared to the configuration in which a table top with casters is used to move the patient as a target, the table 2008 top can be moved more smoothly without shaking the patient too much, and may be prevented from being tangled with a lot of cords of medical equipment and the tubes of medical instruments which run on the floor of the medical room, and may be prevented from being wobbled by stepping over the cords and tubes. Thus, safety and transfer efficiency can be improved.

Examples of the target positions of the robotic table are the same as, or similar to, those described in the first example configuration, and description thereof will be omitted here.

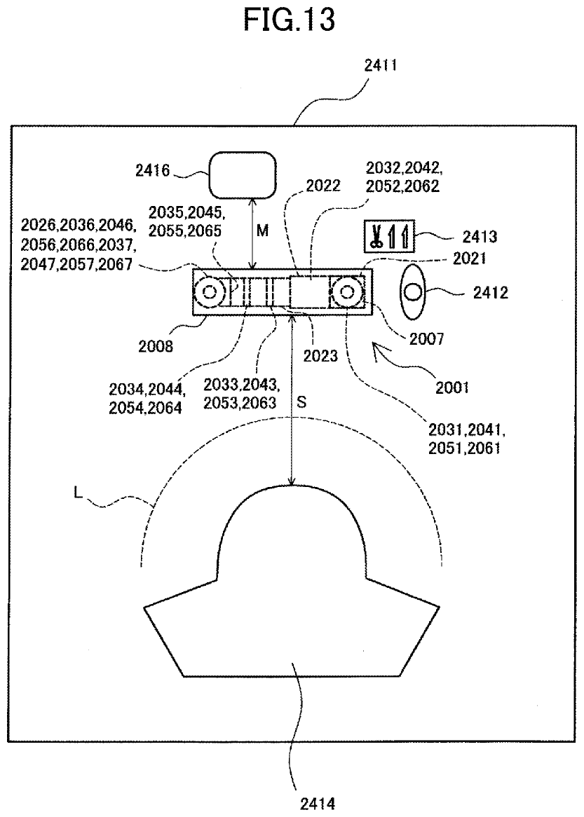

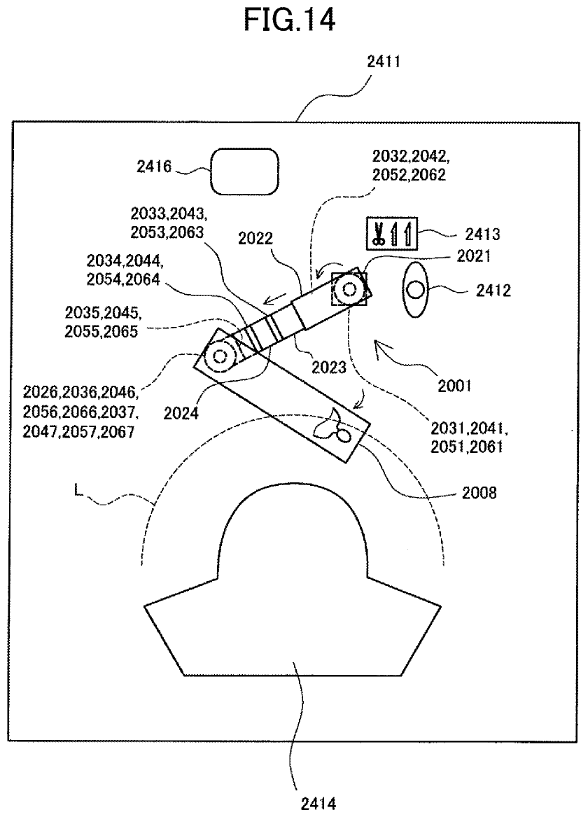

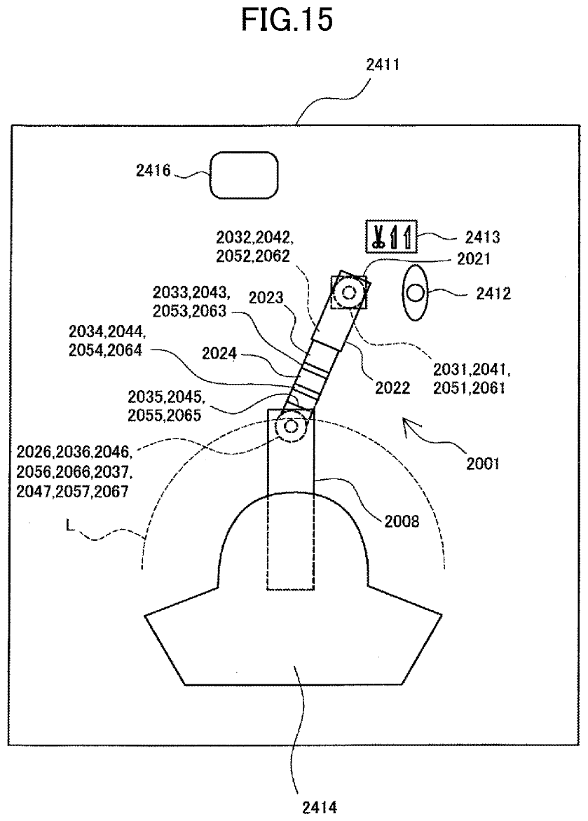

FIG. 13 illustrates a state in which the table top 2008 is located at the placement position (i.e., a first position) in the process of moving a subject, who is a placed target, from the placement position (i.e., the first position) to the inspection position (i.e., a second position) using a robotic table according to the present example configuration. FIG. 14 illustrates a state in which the first movable element 2022, the second movable element 2023, and the table top 2008 are moved by the control of the robot arm controller 2007 as the arrows indicate (in some cases, the height is adjusted by the third movable element 2024, and the table top 2008 is rotated about the fifth axis and/or the sixth axis (the fourth axis and/or the fifth axis in the variation) to have its tilt with respect to the longitudinal direction and/or the width direction of the table top finely adjusted), causing the head of the subject to move toward the inspection device 2414 from an oblique angle. FIG. 15 illustrates a state in which the table top 2008 is inserted in the inspection device 2414, and the subject has arrived at the inspection position. Note that the position of the table top 2008 illustrated in FIG. 13 can also be the treatment position. From the inspection position illustrated in FIG. 15, the respective movable elements move in reverse direction until the table top 2008 returns to the position illustrated in FIG. 13, where a doctor 2412 can give a treatment based on the result of the inspection that has just been conducted.

The robot arm 2001 according to the present example configuration illustrated in FIGS. 9 and 11 has six or seven axes, but is not limited thereto. The robot arm 2001 may have five or six or less axes or may have seven or eight or more axes. Nevertheless, it is preferable that the degrees of freedom of the robot arm be three or more so that the table top 2008 can move at least in a straight manner in the room. For example, omission of the first, fourth, and fifth joints 2031, 2035, and 2036 in FIG. 11 will restrict the movement of the first movable element 2022 in FIG. 14, but it is still possible to move the table top on which a patient lies to the respective target positions.

A joint which travels horizontally straight is used in the present example configuration, which provides an advantage of preventing the movable elements from protruding from the table top in a movement causing the table top to move simply straight, unlike the case of the scalar type in the first example configuration. For example, a ball screw or a rack and pinion mechanism may be employed as a configuration of the joint traveling horizontally straight.

According to the present example configuration, as well, the robot arm can be completely hidden under the table top. However, in some cases, such as when the table top has a shorter length and when the base is more laterally placed to ensure a larger space under the table top, part of the robot arm may not be hidden under the table top, when viewed from vertically above, on any one of the four sides of the table top in the longitudinal direction and the width direction. In terms of space saving, similarly to the first example configuration, the amount of protrusion of the robot arm is preferably less than one fourth (i.e., ¼) of the longitudinal dimension of the table top.

### Third Example Configuration

The robotic table according to the present example configuration is characterized by a slide mechanism provided at the table top of the robotic table in the first and second example configurations.

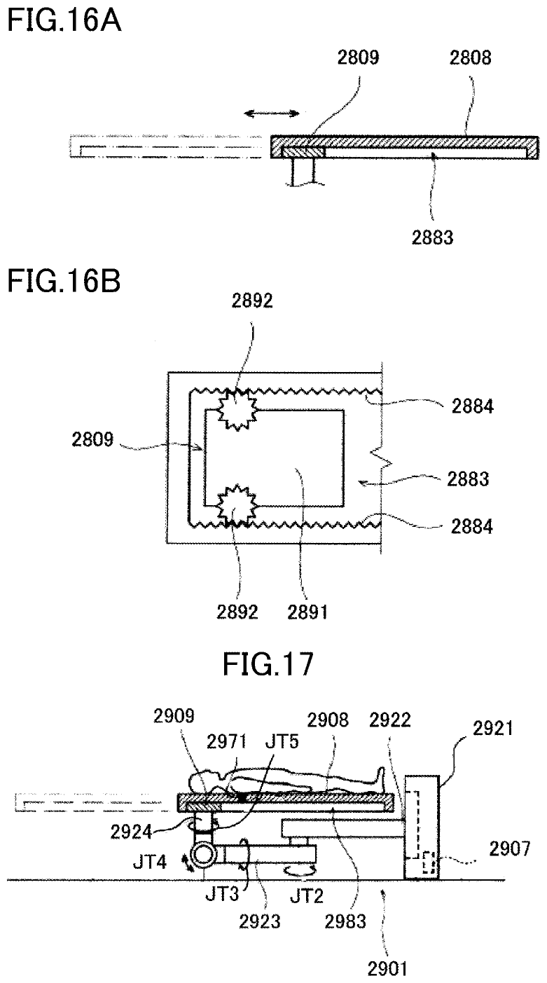

FIG. 16A is a diagram illustrating that the table top 2808 has, in its lower surface, a groove 2883 in which the slide mechanism 2809 is fitted. FIG. 16B is a diagram illustrating that both sides of the groove 2883 are provided with racks 2884 each having a plurality of teeth. The slide mechanism 2809 includes a body 2891 coupled to a distal end of the robot arm, a pair of pinions 2892 movably supported by the body 2891 and engaged with the racks 2884, and an actuator (not shown) which rotates the pinions 2982. If the table top 2808 of the robotic table has this configuration, the table top 2808 may be slid by actuating the actuator to move the placed target farther to an inspection position after the table top has been moved to an inspection preparation position by the robot arm, for example. The actuator may be a servo motor, for example.

Note that by providing the slide mechanism, the degree of freedom in each of the example configurations increments by one. In addition, if the slide mechanism is configured to be driven by the actuator, the actuator of the slide mechanism and the plurality of actuators of the robot arm in the respective example configurations may be actuated simultaneously so that the movable elements of the robot arm and the slide mechanism operate simultaneously to transfer the table top to the target position efficiently.

FIG. 17 is a diagram illustrating a side view of a robotic table according to the first example configuration provided with a slide mechanism. The configurations other than the slide mechanism are the same as those of the first example configuration. The detailed description of the robot arm 2901 is therefore omitted.

Provision of the slide mechanism provides an advantage of downsizing the robot arm, and also an effect that the orientation of the placed target at the placement position (i.e., the first position) is changeable in the first example configuration illustrated in FIG. 6 (in which the robot arm 201 supports one end portion of the table top 208). As for the latter advantage, in the case, for example, where the first position is also a surgery position where surgery of the upper body (e.g., brain or teeth surgery) is performed, the surgeon 612 may have difficulty in performing the surgery if the patient comes back from the inspection device 614 with his/her head directed toward the base 221 as illustrated in FIG. 6, because the base 221 constitutes an obstacle. On the other hand, if the patient comes back from the inspection device 614 with his/her head directed away from the base 221 as illustrated in FIG. 17, it is easy to perform the surgery of the upper body. Since the base 221 does not constitute an obstacle, the surgeon 612 is able to give treatment while seated.

In the examples described herein, the distal end of the robot arm supports the end portion of the table top, but the man-powered slide mechanism may be adopted in a configuration in which the distal end of the robot arm supports a middle portion of the table top. Further, the groove 2883 in the table, in which the actuator-driven slide mechanism body 2909 is fitted, may be provided so as to extend only within the length of the middle portion. In that case, the sliding width decreases, but warpage of the table top is less likely to occur compared to the case of a greater sliding width.

Further, the above examples illustrate the case in which an actuator-driven slide mechanism is employed in the first example configuration. A manually-operated slide mechanism may be employed instead.

[Common Features among Example Configurations]

Additional features applicable to the first to third example configurations will be described below.

(Fixing Member for Tubes/Cords)

If the placed target on the table top in each of the example configurations is a patient, the patient may sometimes be put on a life support system, a drip, or any other equipment necessary for the treatment. For example, the patient is connected to an anesthesia machine 616, 2416 by a tube, for which measures need to be taken in moving the table top.

As described above, compared to the configuration in which a table top with casters is moved, the robotic tables of the first to third example configurations may be prevented from being tangled with such tubes (tubes and/or cables) and from being wobbled by stepping over the tubes during the movement of the placed target. To ensure further safety, it is preferable that the robotic table of one or more embodiments disclosed herein include a fixing member 271, 471, 571, and 2971 attached to at least one of the table top, the base of the robot arm, or the movable element so as to bundle the tubes extending from the equipment mentioned above. This may prevent a situation in which tubes are tangled during the operation of the robot arm more reliably. Moreover, doctors or assistants are prevented from tripping over the tubes, which may further increase the safety. Tubes for which measures to prevent tangles are necessary are not limited to those connected to the equipment, such as a life support system. It is preferable that cords, such as electrical cords for medical equipment and displays, as well, be fixed with the same or similar fixing member. Further, if it is known to which position the table top is to be moved, it is preferable that the movement of the robot arm be roughly predicted to determine how much of the lengths of the tubes/cords should be left unfixed, and where on the tubes/cords the fixing member is to be fitted.

(Configuration of Robot Arm Controller)

As illustrated in FIG. 26, the robot arm controller 207, 407, 507, 2007, and 2907 (hereinafter referred to as “207 to 2907”) is connected to the actuator, the electromagnetic brake, and the position detector of the robot arm 201, 401, 501, 2001, and 2901 (hereinafter referred to as “201 to 2901”).

The robot arm controller 207 to 2907 is connected to an integrated controller 701, and receives a movement instruction from an operating device 705 via the integrated controller 701.

The robot arm controller 207 to 2907 and the integrated controller 701 may be configured as a single controller.

(Table Top Design)

The table top 208, 408, 508, 2008, and 2908 (hereinafter referred to as “208 to 2908”) of each of the above-described example configurations can be appropriately designed according to the circumstances, such as the size of the medical room and a surgical method. Considering the function of the table as a table top, the table top should have a length of at least 210 cm so that a tall patient, for example, can be placed on the table top as a target.

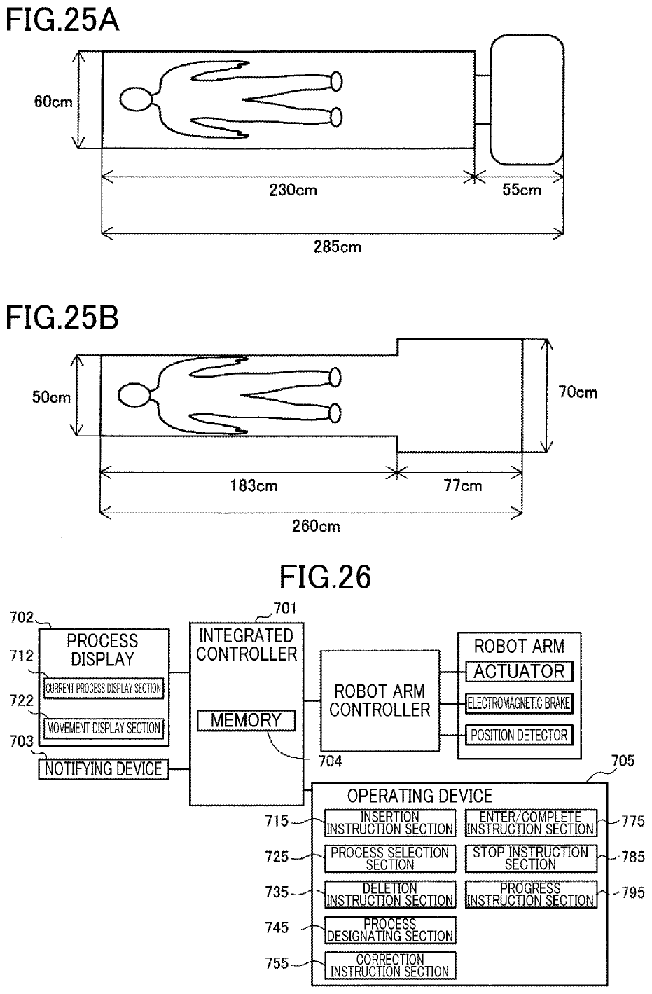

As illustrated in FIG. 25A, if the robotic table is configured to allow the robot arm to protrude from under the table top while the robotic table is taking a position requiring a minimum space in the medical room, it is desirable that the size of the entire robotic table be determined in consideration of the robot arm protruded from under the table top. If the robot arm protrudes in the longitudinal direction of the table top, the longitudinal dimension of the table top should be determined to be shorter than 240 cm because the total length of the robotic table in the space-saving position is desirably at least shorter than 300 cm. Preferably, the protrusion amount of the robot arm from under the table top is one fourth (i.e., ¼) or less of the longitudinal dimension of the table top. Thus, if the longitudinal dimension of the table top is about 240 cm, the maximum allowable dimension of the protrusion of the robot arm from under the table top in the space-saving position is about 60 cm. The table top exemplified in FIG. 25A has a longitudinal dimension of 230 cm. The dimension of the robot arm not hidden under the table top is therefore set to be 55 cm, which is shorter than one fourth (i.e., ¼) of the longitudinal dimension of the table top (i.e., 230 cm). A shorter longitudinal dimension of the table top allows the driving force (i.e., the motor) to have reduced size. Thus, the protrusion amount of the robot arm from under the table top can be slightly reduced.

On the other hand, if the robotic table is configured such that the robot arm is completely hidden under the table top while the robotic table is taking a space-saving position, as illustrated in FIG. 25B, the table top is often required to be larger in size so that the robot arm is housed under the table top, because the robot arm capable of withstanding a load of about 200 kg, for example, needs to be relatively large in size. Thus, the longitudinal dimension of the table top is preferably at least 240 cm, which is longer, for example, than the longitudinal dimension of the table top in the configuration allowing the robot arm to protrude from the table top. Further, it is preferable that the longitudinal dimension of the table top in the configuration allowing the robot arm to be completely hidden under the table top be determined to be shorter than 300 cm because the total length of the robotic table in the space-saving position is desirably shorter than 300 cm. The table top exemplified in FIG. 25B has a longitudinal dimension of 260 cm. In the above description, the length of the table top differs between the case in which the robot arm is completely hidden under the table top and the case in which the robot arm protrudes from under the table top, and is determined based on a certain value, that is 240 cm, as a reference. However, the length of the table top does not need to be determined based on a certain value as a reference. It is not intended to exclude the case in which a range of length values of the table top in one case may overlap with a range of length values of the table top in the other case.

If the width dimension of the table top is too small, it may increase the risk of falling of the patient (i.e., a placed target) from the table top. If the width dimension of the table top is too large, it may serve as an obstacle in reducing the space required. It is therefore preferable to design the width dimension of the table top appropriately according to a customer request. Preferably, the width of the table top is about 45 cm or wider, and shorter than 90 cm not as big as a regular sized single bed. The width of the table top is set to be 60 cm in the example illustrated in FIG. 25A. A T-shaped table top is adopted in the example illustrated in FIG. 25A. The width of the T-shaped table top on one end (a narrow portion) is set to be 50 cm. The width of the T-shaped table top on the other end (a wide portion) is set to be 70 cm. Note that a dimension simply referred to as a “width dimension” in the present specification refers to a maximum width of the table top unless otherwise specified. Note that the table top having a rectangular shape as in FIG. 25A and a table top having a T-shape as in FIG. 25B as viewed from vertically above are useful in bringing a stretcher close to and along a side of the table top and moving the patient (i.e., a placed target) onto the table top.

[Application to Hybrid Operation]

The term “hybrid operation” as used herein refers to carrying out surgery on a patient and capturing of an image of a specific site (an affected area) alternately (at least one back-and-forth movement). The term “hybrid operating room” refers to an operating room equipped with an operation table on which a patient is placed for surgery, and a medical imaging device (i.e., a modality) used to capture an image of a specific site (an affected area). Examples of the medical imaging device include a computerized tomography (CT) scanner, a magnetic resonance imaging (MRI) device, a digital radiographic (DR) imager, a computed radiographic (CR) system, an angiographic X-ray system (an angiographic device, XA), and an ultrasonographic (US) system.

A medical system employing the robotic tables, which have been described so far, as robotic operation tables in a hybrid operation will be described below. The medical system is provided with a base, a robotic operation table configured to support a table top by a robot arm having movable elements coupled together by a joint, and a medical imaging device.

In the medical system which will be described below, the robot arm is capable of moving the table top between a first position where a large portion of the robot arm other than at least the base and the one end portion of the movable element connected to the base is hidden under the table top, when viewed from vertically above, and a second position where at least a portion of the robot arm other than the base and the one end portion of the movable element connected to the base is not hidden under the table top, when viewed from vertically above. The second position is an imaging position where an image is taken by the medical imaging device or an imaging preparation position. The first position is determined to be a position where the shortest distance between the robotic operation table and a location of the medical imaging device at the imaging position or a setback position of the medical imaging device is at least a predetermined distance.

In the hybrid operation, in general, an anesthesia induction process is carried out subsequent to a placement process for placing a patient on the table top. Preferably, an anesthesia induction position of the table top is located at a third position where at least a portion of the robot arm other than the base and the one end portion of the movable element connected to the base is not hidden, when viewed from vertically above, and different from the second position. If the surgery position (the first position) and the anesthesia induction position (the third position) are located at the same position, it is necessary to bring the anesthesia machine closer to the table top during the induction of anesthesia, and set back the anesthesia machine during the surgery to keep a surgery space. In such a situation, it is more efficient to move the patient by using the robotic operation table according to the various example configurations described above, than to move the anesthesia machine. Moreover, it is possible to avoid the risk of falling of the anesthesia machine. Further, the anesthesia induction position is located at the third position because if the anesthesia induction position is located at the same position as the second position (i.e., the imaging position), the imaging device and the anesthesia machine are brought close to each other, casing a situation in which the device or machine not in use during an imaging process or the anesthesia induction process constitutes an obstacle, and adversely affecting the efficiency and the safety.

Note that the patient placement position may be the same as, or different from, the anesthesia induction position. If the placement position is the same as the anesthesia induction position, the transfer process from the placement position to the anesthesia induction position can be omitted. If the placement position is different from the anesthesia induction position, preparations for a first stage of the anesthesia induction process can be made at a place with sufficient space apart from the anesthesia machine.

(Case Using MRI Apparatus as Medical Imaging Device)

The robotic operation tables described above are expected to provide significant effects when used in an intraoperative MRI, in which surgery on an affected area and capturing of an image of the affected area by the MRI apparatus are alternately carried out (at least one back-and-forth movement). In the intraoperative MRI for removing brain tumors, the number of times of moving the patient and imaging his/her brain with the MRI apparatus is defined to be two to four, and three on average (see “JIYUKUKAN” Vol. 25, Appendix of “Front-line system for total removal of brain tumor which allows increasing survival rate and ensuring postoperative QOL,” Hitachi Medical Corporation, INNERVISION, September (2012)). Thus, there is a high need for moving the patient back and forth between the imaging position, where images are taken by the MRI apparatus, and the treatment position accurately and quickly during surgery.

Described below is a technique for applying the robotic operation tables as the robotic tables having the first to third example configurations (in some cases, the robotic operation tables with the above-described common additional features) to the intraoperative MRI, in which images of a specific site of a patient as a placed target are taken by an MRI apparatus, and thereafter the patient is moved to a surgery position where surgery is performed immediately.

In the following description, it will be described, with reference to the drawings, how the table top 208 to 2908 is moved between the surgery position and the MRI scanning position by actuating the robot arm 201 to 2901.

If the robotic tables of the respective example configurations are applied to the intraoperative MRI, the apparatuses 614 and 2414 placed in the operating room in the description of the movement of the table top having the respective example configurations are MRI apparatuses.

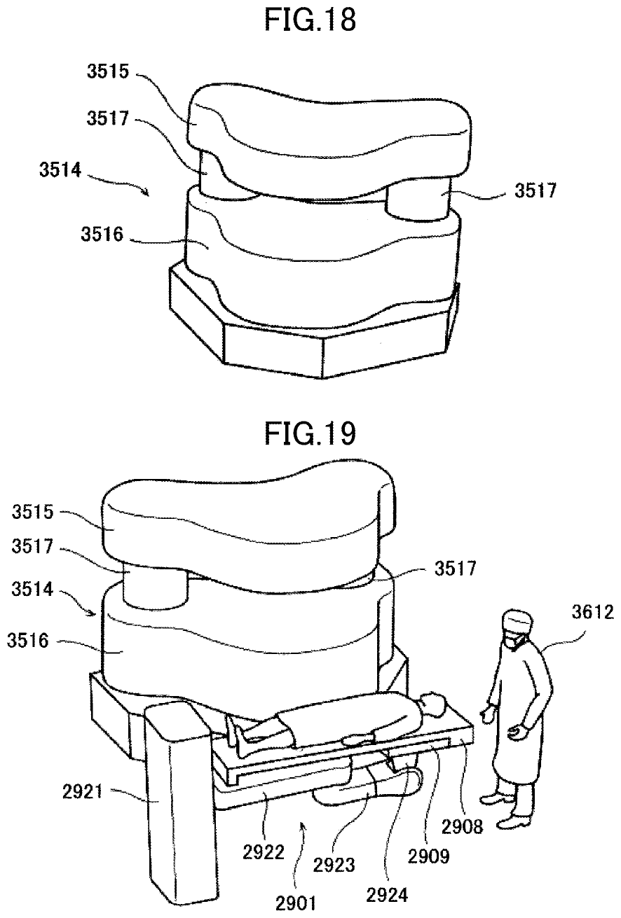

FIG. 18 illustrates an open MRI apparatus 3514. The open MRI apparatus 3514 is open at the front and lateral sides. Specifically, the open MRI apparatus 3514 includes an upper inspection section (an upper magnet) 3515 and a lower inspection section (a lower magnet) 3516, each of which is in an approximately T-shape with its middle portion protruding forward. Formed between these inspection sections 3515 and 3516 is a space in which the table top, where the patient is placed, is to be inserted. The upper and lower inspection sections 3515 and 3516 are coupled together by a pair of support columns 3517 at their respective end portions. The MRI apparatus 3514 may also be a donut-shaped MRI apparatus. However, if the donut-shaped MRI apparatus 3514 is applied to a case (e.g., the case illustrated in FIG. 7) in which the patient is easily inserted in the MRI apparatus from an oblique angle, the table top needs to be positioned in front of the hollow of the donut before being inserted into the hollow, which may make the movement of the robot arm a little less flexible.

The space defined between the upper inspection section (i.e., the upper magnet) 3515 and the lower inspection section (i.e., the lower magnet) 3516 is an imaging space. It can be said that the table top 208 to 2908 is in the MRI scanning position when at least part of the table top 208 to 2908 overlaps with this imaging space. The position of the table top 208 to 2908 in the imaging space is not always the same, and differs depending on a site to be imaged of the patient and the height and size of the patient. However, a particular position in the imaging space can be stored in a memory in the robot arm controller. In a typical hybrid operation, the table top moves back and forth between the surgery position and the imaging position multiple times. Thus, the imaging position and/or the surgery position for each surgery may be stored in the memory.

FIG. 6 illustrates a state in which the table top 208 on which the patient is to be placed is located at the surgery position (i.e., the first position) in the process of moving the table top 208 from the surgery position (i.e., the first position) to the MRI scanning position (i.e., the second position) using the robotic table according to the first example configuration as a robotic operation table. As illustrated in FIG. 6, since the table top 208 is located at the first position, portions of the robot arm 201 which are the base and the one end portion of the movable element connected to the base are not hidden under the table top 208 on one of the longitudinal ends of the table top 208, and the rest of the robot arm 201 is hidden under the table top 208, when the table top 208 is viewed from vertically from above. The maximum dimension of the robot arm 201 not hidden under the table top 208 is less than one fourth (i.e., ¼) of the longitudinal dimension of the table top 208.

FIG. 7 illustrates a state in which the second movable element 223 and the table top 208 are moved by the control of the robot arm controller 207 as the arrows indicate (in some cases, the first movable element 222, too, is moved in the vertical direction to have its height adjusted, and the table top 208 is rotated about the third axis and/or the fourth axis to have its tilt with respect to the longitudinal direction and/or the width direction of the table top finely adjusted), causing the head of the patient to move toward the MRI apparatus 614 from an oblique angle. FIG. 8 illustrates a state in which one end portion of the table top 208 is inserted in the MRI apparatus 614, and the patient has arrived at the MRI scanning position which is the second position. As illustrated in FIG. 8, of the robot arm 201, the entire movable element 222 directly connected to the base 221, and one end portion of the movable element 223 not directly connected to the base 221, are not hidden under the table top 208. The maximum dimension of the robot arm 201 not hidden under the table top 208 is at least one fourth (i.e., ¼) of the longitudinal dimension of the table top 208.

If the table top 208 needs to be moved to the surgery position after the capturing of images by the MRI apparatus 614 so that the surgeon 612 can perform surgery on the patient, the respective movable elements are controlled by the robot arm controller 207 to cause the table top 208 to move in the reverse direction from the MRI scanning position (i.e., the second position) illustrated in FIG. 8 to the surgery position (i.e., the first position) illustrated in FIG. 6. The table top 208 returns to the surgery position in this manner, where the surgeon 612 can immediately start appropriate surgery while viewing the images taken by the MRI apparatus 614.

Now, the case in which the table top 208 is transferred also to the anesthesia induction position (i.e., the third position) in addition to the surgery position (i.e., the first position) and the imaging position (i.e., the second position) will be described.

In the intraoperative MRI, in general, an anesthesia induction process is carried out subsequent to a placement process for placing a patient on the table top. Note that the patient placement position may be the same as, or different from, the anesthesia induction position.

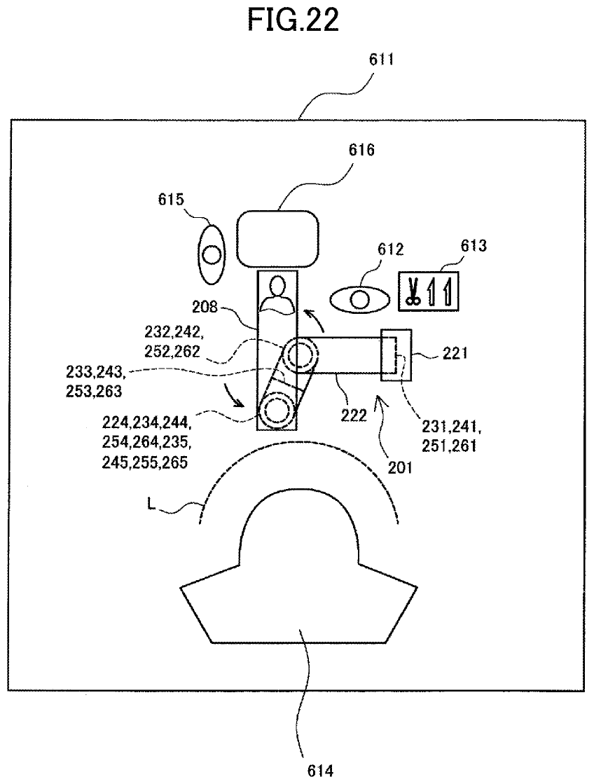

FIG. 22 illustrates the case in which the patient placement position is different from the anesthesia induction position, and the same as the surgery position. In FIG. 22, the table top 208 is transferred from the placement position located at the first position to the anesthesia induction position located at the third position.

After the patient is placed on the table top 208 at the first position, the second and fifth joints 232 and 235 are rotated (in some cases, the height of the table top 208 is adjusted by the first joint 231, and the tilt of the table top 208 with respect to the longitudinal direction and/or the width direction of the table top 208 is adjusted by the third and/or fourth joint 233, 234) to cause the table top 208 to move as the arrows indicate in FIG. 22 to a position where one end of the table top 208 is close to the anesthesia machine 616. The distance between the anesthesia machine and the table top at this position close to the anesthesia machine is about 10 cm to 40 cm (although the distance may vary depending on the location of the patient on the table top), considering that an anesthesiologist uses one hand to put a mask or the like provided at the end of tube on the patient's mouth, and the other hand to handle a pump of the anesthesia machine. At the anesthesia induction position (i.e., the third position) illustrated in FIG. 22, the base 221 and the movable element 222 directly connected to the base 221 are not hidden under the table top 208, when the table top 208 is viewed from vertically above. The maximum dimension of the robot arm 201 not hidden under the table top 208 is at least one fourth (i.e., ¼) of the longitudinal dimension of the table top 208. Note that this transfer process is omitted if the placement position is the same as the anesthesia induction position.

The anesthesiologist 615 then gives anesthesia to the patient. After the completion of the anesthesia, the respective movable elements are moved to cause the table top 208 to move in the direction opposite to the direction of the arrows illustrated in FIG. 22, so that the table top 208 is transferred to the surgery position, which is the first position. The surgeon 612 performs surgery on the patient based on the information of the image captured by the MRI apparatus prior to the surgery. After brain tumors, for example, are removed, the table top 208 is transferred to the imaging position, which is the second position, as described above, where the affected area (e.g., brain) is subjected to MRI. The table top 208 is then transferred back to the surgery position located at the first position. If there is a remaining tumor, for example, the surgeon 612 continues to perform the surgery.

FIG. 13 illustrates a state in which the table top 2008 on which the patient is to be placed is located at the surgery position (i.e., the first position) in the process of moving the table top 2008 from the surgery position (i.e., the first position) to the MRI scanning position (i.e., the second position) using the robotic table according to the second example configuration as a robotic operation table. As illustrated in FIG. 13, since the table top 2008 is located at the first position, the entire robot arm 2001 is hidden under the table top 2008 when the table top 2008 is viewed from vertically above.

FIG. 14 illustrates a state in which the first and second movable elements 2022 and 2023 and the table top 2008 are moved by the control of the robot arm controller 2007 as the arrows indicate (in some cases, the third movable element 2024, too, is rotated about the third axis to have its height adjusted, and the table top 2008 is rotated about the fifth axis and/or the sixth axis to have its tilt with respect to the longitudinal direction and/or the width direction finely adjusted), causing the head of the patient to move toward the MRI apparatus 2414 from an oblique angle. FIG. 15 illustrates a state in which the table top 2008 is inserted in the MRI apparatus 2414, and the table top 2008 has arrived at the MRI scanning position. As illustrated in FIG. 15, of the robot arm 2001, the entire movable element 2022 directly connected to the base 2021, and the second movable element 2023 and other elements not directly connected to the base 2021, are not hidden under the table top 2008 at the imaging position, which is the second position. The maximum dimension of the robot arm 2001 not hidden under the table top 2008 is at least one fourth (i.e., ¼) of the longitudinal dimension of the table top 2008.

If the table top 2008 needs to be moved to the surgery position after the capturing of images by the MRI apparatus 2414 so that the surgeon 2412 can perform surgery on the patient, the respective movable elements are controlled by the robot arm controller 2007 to cause the table top 2008 to move in the reverse direction from the MRI scanning position (i.e., the second position) illustrated in FIG. 15 to the surgery position (i.e., the first position) illustrated in FIG. 13. The table top 2008 returns to the surgery position in this manner, where the surgeon 2412 can immediately start appropriate surgery while viewing the images taken by the MRI apparatus.

Similarly to the case using the robotic table according to the first configuration, the table top 2008 of the second configuration, too, is capable of moving to the anesthesia induction position, which is the third position.

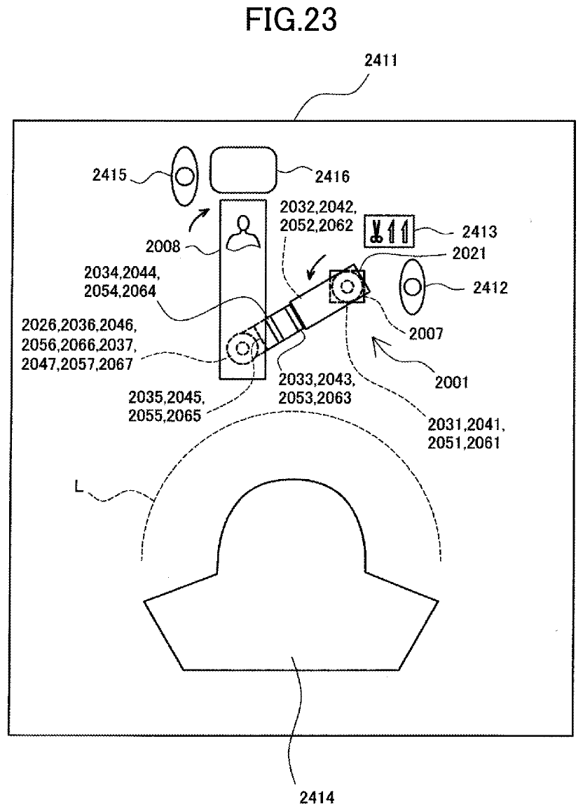

FIG. 23 illustrates the case in which the patient placement position is different from the anesthesia induction position, and the same as the surgery position. In FIG. 23, the table top 2008 is transferred from the placement position located at the first position to the anesthesia induction position located at the third position.

After the patient is placed on the table top 2008 at the first position, the first and seventh joints 2031 and 2037 are rotated (in some cases; the height of the table top 2008 is adjusted by the third and fourth joints 2033 and 2034; the distance from the base 2021 to the sixth axis is adjusted by the second joint 2032; and the tilt of the table top 2008 with respect to the longitudinal direction and/or the width direction of the table top 2008 is adjusted by the fifth and/or sixth joint 2035, 2036) to cause the table top 2008 to move as the arrows indicate in FIG. 23 to a position where one end of the table top 2008 is close to the anesthesia machine 2416. At the anesthesia induction position (i.e., the third position) illustrated in FIG. 23, the base 2021 and the movable element 2032 directly connected to the base 2021 are not hidden under the table top 2008, when the table top 2008 is viewed from vertically above. The maximum dimension of the robot arm 2001 not hidden under the table top 2008 is at least one fourth (i.e., ¼) of the longitudinal dimension of the table top 2008. Note that this transfer process is omitted if the placement position is the same as the anesthesia induction position.

The anesthesiologist 2415 then gives anesthesia to the patient. After the completion of the anesthesia, the respective movable elements are moved by the control of the robot arm controller 2007 to cause the table top 2008 to move in the direction opposite to the direction of the arrows illustrated in FIG. 23, so that the table top 2008 is transferred to the surgery position, which is the first position. The surgeon 2412 performs surgery on the patient based on the information of the image captured by the MRI apparatus prior to the surgery. After brain tumors, for example, are removed, the table top 2008 is transferred to the imaging position, which is the second position, as described above, where the affected area (e.g., brain) is subjected to MRI. The table top 2008 is then transferred back to the surgery position located at the first position. If there is a remaining tumor, for example, the surgeon 2412 continues to perform the surgery.

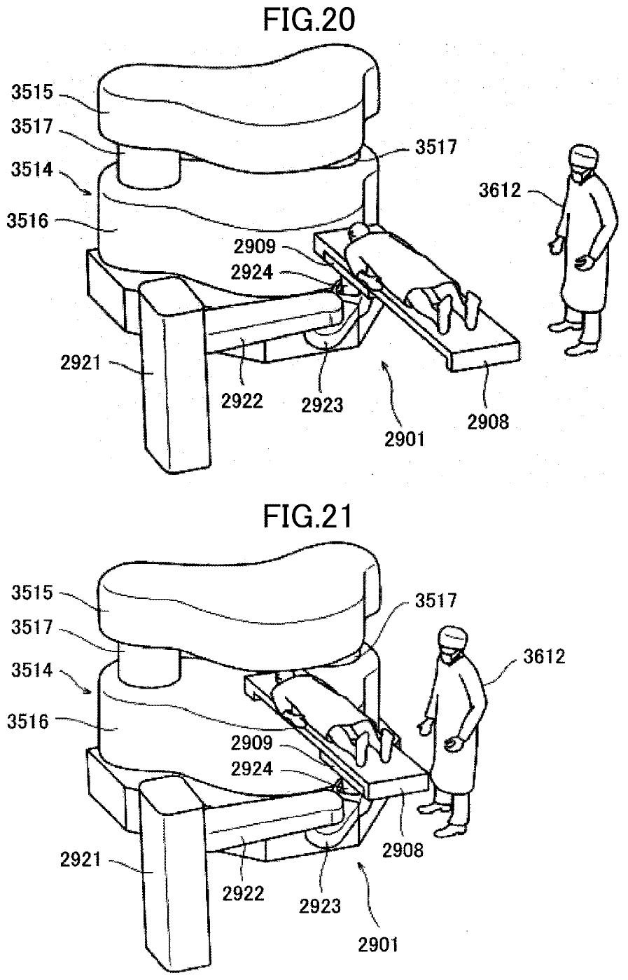

FIGS. 19 to 21 are perspective diagrams for illustrating how the robotic operation table according to the third example configuration (which employs an actuator-driven slide mechanism in the robotic table of the first example configuration) moves when used in the intraoperative MRI. In FIG. 19, the table top 2908 is located at the first position, which is the patient placement position and is also the surgery position. The second movable element 2923 makes a horizontal rotation about the second axis, and at the same time the table top 2908 makes an axial rotation about the fifth axis (in some cases, the height of the table top 2908 is adjusted by the first joint, and the tilt of the table top 2908 with respect to the longitudinal direction and/or the width direction of the table top 2908 is adjusted by the third and/or the fourth joint), causing the table top 2908 to move to the MRI preparation position illustrated in FIG. 20. Then the table top 2908 is slid, by the actuation of the actuator, to a position where the table top 2908 overlaps with the imaging space of the MRI apparatus, and moved to the MRI scanning position, which is the second position (FIG. 21).

The robotic table according to the third example configuration includes a slide mechanism, which may avoid an increase in the length of the first and second movable elements for ensuring a wide range of movement of the table top. Thus, provision of the slide mechanism provides an advantage of downsizing the robot arm, and also an effect that the orientation of the head of the patient at the surgery position (i.e., the first position) is changeable in the robotic table according to the first example configuration illustrated in FIG. 2 in which the robot arm 201 supports one end portion of the table top 208. As for the latter advantage, in the case, for example, where the intraoperative MRI is used to perform surgery relating to the upper body (e.g., removal of brain tumors), the surgeon 612 may have difficulty in performing the surgery if the patient on the table top 208 comes back from the MRI apparatus 614 with his/her head directed toward the base 221 as illustrated in FIG. 2, because the base 221 constitutes an obstacle. On the other hand, if the patient on the table top 2908 comes back from the MRI scanning position with his/her head directed away from the base 2921 as illustrated in FIG. 19, it is easy to perform the surgery of the upper body, such as the head. Since the base 2921 does not constitute an obstacle on the side close to the upper body during the surgery, the surgeon 3012 may lower the height of the table top 2908 and give treatment while seated.

The MRI preparation position illustrated in FIG. 20 is a position where the table top 2908 does not overlap with the imaging space, where the particular direction (i.e., the longitudinal direction) of the table top 2908 is directed to the opening of the MRI apparatus 3314 when the table top 2908 is located close to the imaging position (e.g., 10 cm to 40 cm from the imaging space), and where the table top is parallel to the particular direction (i.e., the longitudinal direction) of the table top at the imaging position. In the case of the open MRI apparatus, which has a wide opening, the table top may be directed to the MRI apparatus in a plurality of different directions. In the case of the donut-shaped MRI apparatus, the direction of the table top toward the opening is substantially uniquely determined. The movement of the table top 2908 may be stopped for a while at this imaging preparation position, where an assistant, for example, may prepare for the MRI (e.g., check if there is any metallic object, and correct the position and posture of the patient), and thereafter the table top 2908 may be transferred to the MRI apparatus. Of course, the table top may just pass through the MRI preparation position without stopping there for a while, and smoothly move to the MRI scanning position.

The surgery position as the first position described above is located at a position where the table top is not close to the imaging space, that is, a position at least a predetermined distance away from the imaging space. In the above examples, a surgical instrument table 613 and 2413 on which surgical instruments to be used by the surgeon 612, 2412, and 3612 are placed, is disposed near the surgery position. If these surgical instruments are placed close to the MRI apparatus, the surgical instruments may be affected (e.g., may float) by the permanent magnet of the MRI apparatus, and may hurt the patient and those who handle the surgical instruments. It is therefore preferable that the treatment position be sufficiently away from the MRI apparatus, preferably farther away from the 5 Gauss line L.

It is also preferable that the base 221, 421, 521, 2021, and 2921 (hereinafter referred to as “221 to 2921”) of the robot arm be located outside the 5 Gauss line L. The base 221 to 2921 of the robot arm is provided with a big motor, which includes a magnet. If this motor is located close to the MRI apparatus, the magnetic field generated at the imaging space of the MRI apparatus is distorted, which leads to a deterioration of the MRI images.

Thus, it is preferable that the robotic table comprised of the robot arm and the table top be configured such that the surgery position, which is the first position, is determined to be a position where a shortest distance S to the MRI apparatus is at least a predetermined distance. Considering the safety, the shortest distance S is preferably set to be 5 Gauss line L.

Regarding 5 Gauss line, low magnetic field MRI apparatuses have been developed, which, for example, have the static magnetic field strength of 0.3 Tesla and allow the 5 Gauss line to be set at about one meter from the gantry edge (see, “Intelligent Operating Theater and MR-compatible Operating System” MEDIX, 39: 11-16, 2001). Thus, the shortest distance S between the MRI apparatus and the robotic table located at the first position is preferably at least 1 m. The shortest distance S may be reduced a little, depending on the development of the low magnetic field MRI apparatuses.

The shortest distance S is preferably at least 1.5 m, for example, in order to use an MRI apparatus with a larger magnetic field or ensure a further improvement in safety.

However, if the treatment position, which is the first position, is away from the MRI apparatus, a large robot arm capable of withstanding a heavy load needs to be used so that the table top can be transferred to the imaging position, which is the second position, considering, for example, the load of the table top supported by the robot arm. In the case of using a large robot arm, it is difficult for a large portion of the robot arm to be housed under the table top at the surgery position located at the first position (which means that the robot arm constitutes an obstacle while the surgeon and assistants surround the table top to carry out surgery). In addition, the increased distance from the MRI apparatus to the robotic table requires an operating room to be increased in size accordingly. It is therefore not that the greater shortest distance S between the MRI apparatus and the robotic table at the first position, the better.

The first position of the robotic table is better close to the MRI apparatus as long as sufficient safety can be ensured in a relationship between the robotic table and the MRI apparatus. For example, in the case of a 1.5 Tesla MRI apparatus, the 5 Gauss line is about 2.8 m from the gantry (i.e., the MRI apparatus) at the shortest distance (see, “Avoid attraction accident of 3T MRI” Toshio Tsuchihashi, INNERVISION, September (2012)). Considering the 5 Gauss line, the rigidity of the robot arm (i.e., stability of the table top) and downsizing, the maximum shortest distance S between the MRI apparatus and the robotic table located at the first position is preferably set to be 3 m or less, for example. In the case of the MRI apparatus having a static magnetic field strength of 0.3 Tesla and the 5 Gauss line of about 1 m, the maximum shortest distance S may be about 2 m, considering a situation in which a person gripping a surgical instrument may stand by the MRI apparatus.

As is also described in the “Avoid attraction accident of 3T MRI” (INNERVISION, September (2012)), the 5 Gauss line forms an oval around the MRI apparatus, and is 2.8 m at the shortest distance, and 5 m at the longest distance, from the MRI apparatus in the case of a 1.5 Tesla MRI apparatus. In most cases today, an operation table capable of rotating and elevating and having a slidable top plate is employed in the intraoperative MRI. If the operation table can only make these three types of movement, the position of the operation table which allows the table top to move toward the imaging position may be limited, resulting in difficulties in installing the operation table top near the shortest distance portion of the 5 Gauss line. The robotic table, on the other hand, provides a high degree of freedom in determining the transferring direction of the table top, as described above, and hence a high degree of freedom in determining the place of installment, as well.

The anesthesia induction position, which is the third position, is preferably located opposite to the MRI apparatus in the width direction of the table top (i.e., the direction orthogonal to the longitudinal direction of the table top) when the table top is located at the surgery position (i.e., the first position). This is because it is preferable that the anesthesia machine, which does not require mobility in principle, be placed at a different position from a position between the surgery position (i.e., the first position) and the imaging position (i.e., the second position) in the intraoperative MRI in which the table top is moved back and forth between the surgery position (i.e., the first position) and the imaging position (i.e., the second position). The shortest distance M between the surgery position (i.e., the first position) and the anesthesia induction position (i.e., the third position) is preferably at least 80 cm, so that medical equipment (e.g., a surgical microscope) can be placed around the table top during surgery. For example, the base portion of the surgical microscope OME-9000 manufactured by Olympus Corporation has a diameter of 80 cm. Thus, if the shortest distance M between the surgery position (i.e., the first position) and the anesthesia induction position (i.e., the third position) is at least 80 cm, the surgical microscope can be placed around the table top without moving the anesthesia machine.

In the case of employing a movable MRI apparatus in a system configuration, the above-described shortest distances and the first to third positions are determined depending on whether the MRI apparatus is moved or fixed during the surgery. For example, if the MRI apparatus is moved from a next room and fixed during the surgery, the shortest distance S may be determined in relation to the fixed position during the surgery. If the MRI apparatus is moved to a particular position only for the purpose of capturing images, and moved back to the setback position after the image capturing, the shortest distance S may be determined in relation to the setback position of the MRI apparatus.

As can be seen from the foregoing description, application of the robotic tables having the first to third example configurations to the robotic operation tables for the intraoperative MRI allows the patient placed on the table top to be moved between the surgery position (i.e., the first position) and the MRI scanning position (i.e., the second position) quickly and accurately by the operation of the robot arm. This structure may contribute to enhancing the superior effect of improving the performance of surgery. According to the aforementioned document, “JIYUKUKAN” Vol. 25, Appendix of “Front-line system for total removal of brain tumor which allows increasing survival rate and ensuring postoperative QOL,” Hitachi Medical Corporation, INNERVISION, September (2012), compared to the conventional brain tumor removal surgery in which the MRI and surgery have been performed in different rooms, application of the intraoperative MRI in which imaging and surgery are performed in the same room (and further application of information-guided surgery) achieves five-year survival rates of 78% in grade 3 and 19% in grade 4, which are about three times the average conventional five-year survival rates of about 25% in grade 3 and about 7% in grade 4 of the surgery performed in different rooms. Application of the robotic tables having the first to third example configurations to the intraoperative MRI allows the table top and the patient to be transferred quickly and accurately as described so far, and allows the MRI scanning and the brain tumor removal surgery to be performed efficiently. Also, these robotic tables are highly expected to contribute to further improving the survival rate. In particular, as explained earlier, in the brain tumor removal surgery, the MRI scanning and the brain tumor removal surgery are not performed only once, but are repeated several times. Thus, there are high expectations for the quick and accurate transfer of the patient between the treatment position and the MRI scanning position.

In applying the robotic tables of the first to third example configurations to the intraoperative MRI, it is preferable that the supply of a drive current to the plurality of actuators mounted on the robot arm 201 to 2901 be stopped and the brake functions of the plurality of electromagnetic brakes associated with the actuators be turned on by the control of the robot arm controller 207 to 2907, during a period after the table top 208 to 2908 has arrived at the MRI scanning position 614, 2414, and 3514 and before images of the target placed on the table top starts to be taken. This configuration is intended to reduce the deterioration of the MRI images due to effects of the magnetic field generated while the actuators are actuated, for the MRI apparatus takes images by utilizing the static magnetic field. This control may be automatically carried out when the controller detects that the table top has arrived at the MRI scanning position and stayed there for a predetermined period of time, or may be carried out in accordance with a manually entered instruction. It is preferable, however, that the start of MRI scanning (e.g., at a time when the main power of the MRI apparatus is turned on, or the MRI apparatus is turned into an active state) trigger the checking of whether the actuators of the robot arm are actuated or not. If the actuators are actuated, the actuators are forcedly turned off to have the brake functions turned on. It is therefore preferable that the robot arm controller 207 to 2907 have an MRI operation monitor to monitor, for example, whether the main power of the MRI apparatus is turned on or whether the MRI apparatus is turned into an active state.

In some cases, the robot arm of the third example configuration may be provided with a man-powered slide mechanism. Thus, the supply of the drive current to the plurality of actuators mounted on the robot arm 201 to 2901 may be stopped and the brake functions of the plurality of electromagnetic brakes associated with the actuators may be turned on by the control of the robot arm controller 207 to 2907 at a time when the table top 208 to 2908 arrives at the MRI preparation position. After the actuators are turned off and the brake functions of the electromagnetic brakes are turned on, the slide plate is slid to move the patient to the MRI scanning position.

The table top may be moved between the surgery position and the MRI scanning position by actuating the robot arm 201 to 2901 through a teaching pendant as an operating device. However, if the surgery position and the MRI scanning position are stored in advance in the robot arm controller 201 to 2907, the table top 208 to 2908 may move between the surgery position and the MRI scanning position more quickly and smoothly according to a movement control program for the table top 208 to 2908 with respect to the first, second and/or third position. For example, if the table top is configured to move according to this movement control program only during a forward-movement instruction given through a teaching pendant, safety is ensured because the execution of the program is interrupted by stopping the forward-movement instruction (e.g., stopping pushing the button).

In the case where the robot arm automatically transfers the table top between the surgery position and the MRI scanning position, it is the accuracy of the positioning of the robot arm that brings the surgical field back to exactly where it used to be after the MRI scanning. Another advantage of using the robot arm is that it is possible to ensure a wide surgical field during surgery by operating the robot arm and changing the position and posture of the patient during the surgery.

(Case Using Apparatus Other than MRI Apparatus as Medical Imaging Device)

In the case in which an apparatus other than the MRI apparatus is used as the medical imaging device, a system design is slightly different from the case of the intraoperative MRI in that it is not necessary to take measures against the magnetic field in introducing the robotic operation table as a robotic table. However, table top movements, for example, are basically the same as those in the case in which the MRI apparatus is used as the medical imaging device.

If an apparatus other than the MRI apparatus is used as the medical imaging device, the device 614 in FIGS. 6 to 8, and the device 2414 in FIGS. 13 to 15, which are referred to in describing the movement of the table top in the respective example configurations, are angiographic devices, for example. In FIGS. 6 and 13, the table top 208 to 2008 are each located at the surgery position, which is the first position. Similarly to the case in which the MRI apparatus is used as the medical imaging device, the respective movable elements and the table top 208 to 2008 are moved in the directions indicated by the arrows shown in FIGS. 7 and 14, causing the table top 208 to 2008 to reach at the imaging position (i.e., the second position) illustrated in FIGS. 8 and 15.

The imaging position and the imaging preparation position may be the same as those in the case where the MRI apparatus is used as the medical imaging device. That is, it can be said that the table top 208 to 2008 is located at the imaging position, where images are taken by the medical imaging device, when at least part of the table top 208 to 2008 overlaps with the imaging space of the medical imaging device. In the case where the medical imaging device is an angiographic device, the imaging space is defined by an X-ray tube (i.e., an X-ray irradiation side) and an imaging system (i.e., an X-ray receiving side). The imaging preparation position is a place where the table top 208 to 2008 is close to the imaging space, but does not overlap with the imaging space.

Examples of the anesthesia induction position, which is the third position, are illustrated in FIGS. 22 and 23. In the case where the placement position and the surgery position are located at the same first position, the positions illustrated in these figures are where the table top has reached at the anesthesia induction position. In the case where the placement position and the anesthesia induction position are located at the same third position, the positions illustrated in these figures are the placement position and the anesthesia induction position. Similarly to the case in which the MRI apparatus is used as the medical imaging device, the anesthesia induction position (i.e., the third position) is preferably located opposite to the angiographic device with respect to the table top width direction when the table top is located at the first position.

At the imaging position, which is the second position, images of a specific site (an affected area) of the patient is taken by X-ray fluoroscopy using a single-plane or biplane angiographic device. Then, the table top 208 to 2008 is moved to the surgery position (i.e., the first position) to give catheter treatment or any other treatment.

Examples of the angiographic device includes a ceiling traveling type in which the angiographic device is suspended from the ceiling and travels along the rail provided on the ceiling, a floor-fixed type in which a support of the device is fixed to the floor such that the body (i.e., a C-shaped portion) of the device is rotatable about a vertical axis, and a floor traveling type in which a support of the device is provided with casters, and the device as a whole can travel on the floor on the casters. Taking images by any one of these angiographic devices is called single-plane imaging. Bidirectionally performing fluoroscopy and imaging at one time, using two angiographic devices (e.g., the ceiling traveling type and the floor-fixed type) in combination, is called biplane imaging. The biplane system is widely used due to its effects of reducing a burden on the patient, that is, reducing imaging time, exposure dose, and the amount of a contrast agent to be used.

Regardless of whether the single-plane system or multi-plane system is used, the first to third positions are determined on the same basis.

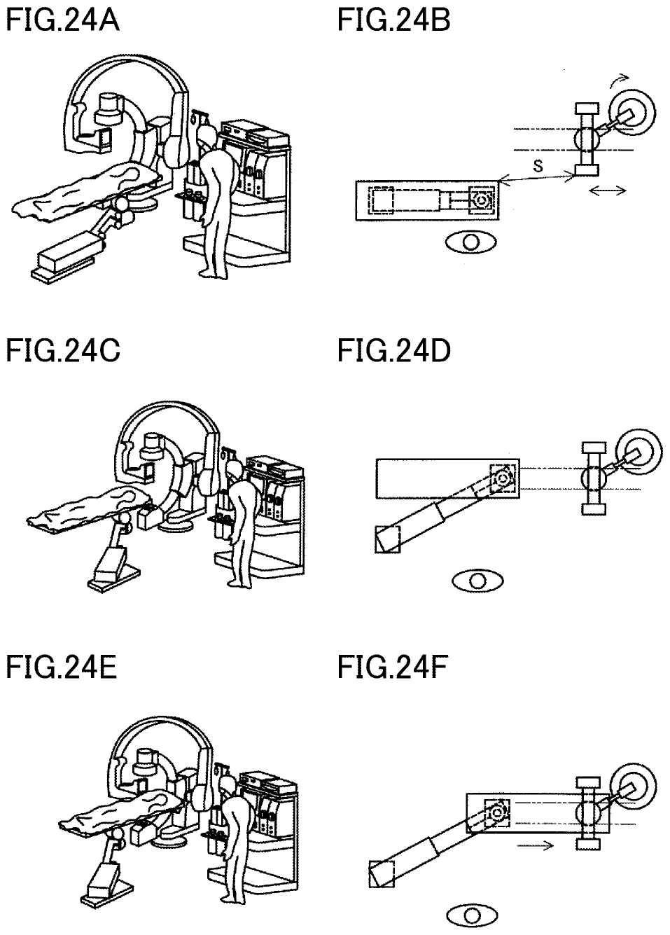

FIGS. 24A to 24F illustrate an example in which a biplane angiographic device comprised of a combination of the ceiling traveling type and the floor-fixed type is used as the medical imaging device. In this example, a robotic table having the second example configuration (illustrated in FIG. 11) and equipped with a slide mechanism is used as a robotic operation table to move the table top from the surgery position (i.e., the first position) to the imaging position (i.e., the imaging position). FIGS. 24A, 24C, and 24E are perspective views. FIGS. 24B, 24D, and 24F are plan views of the operating room as viewed from vertically above.

In FIGS. 24A and 24B, the table top is located at the surgery position, which is the first position, where the entire robot arm is hidden under the table top. In FIGS. 24C and 24D, the first and sixth joints are rotated, and the second joint is extended and retracted to adjust the distance between the sixth joint and the base (in some cases, the third joint is rotated to adjust the height of the table top, and the tilt of the table top with respect to the longitudinal direction and/or the width direction of the table top is adjusted by the fourth and/or fifth joint), causing the table top to reach at the imaging preparation position. In FIGS. 24E and 24F, the table top has reached at the imaging position (i.e., the second position) by the actuation of the slide mechanism.

How the first position is determined in the case where the angiographic device is used as the medical imaging device is similar to how the first position is determined in the case where the MRI apparatus is used as the medical imaging device. The first position is determined in consideration of the shortest distance S between the angiographic device and the robotic table at the surgery position (i.e., the first position). In the case where the angiographic device is used as the medical imaging device, it is not necessary to take the 5 Gauss line into account because it is not necessary to take the effect of magnetic properties into account. However, the shortest distance S is preferably set to be at least a predetermined distance away from the angiographic device when the table top is located at the surgery position (i.e., the first position) so that the surgeon and the assistants may surround the table top. In the hybrid operation, too, using the angiographic device as the medical imaging device, the shortest distance S is preferably set to be at least a predetermined distance away from the angiographic device so that medical equipment (e.g., a surgical microscope) can be placed around the table top during surgery. The shortest distance S may be at least 80 cm, considering, for example, the diameter of the base portion of the surgical microscope, so that the surgical microscope can be placed between the robotic operation table and the angiographic device.

Further, similarly to the case using the MRI apparatus as medical imaging device, it is not that the greater shortest distance S between the angiographic device and the robotic operation table at the first position, the better, considering the load of the table top supported by the robot arm, how much the robot arm located at the surgery position (i.e., the first position) can be stored under the table top (that is, downsizing of the robot configuration), and the rigidity of the robot arm (that is, stability of the table top). Thus, the shortest distance S between the angiographic device and the robotic operation table at the first position is preferably set to be, for example, 80 cm to allow the surgical microscope to be installed, and further to be 2 m or less, considering a space to allow a person to pass therethrough.

In the case where the medical imaging device is a ceiling traveling type or floor-fixed type angiographic device, the device can move back and forth between the imaging position and the setback position by moving the device along the rail, or rotating the body (the C-shaped portion) of the device with respect to the support of the device, even during the surgery.

Unlike the robotic operation table used only for image taking by a medical imaging device, the above-described robotic operation table used in a hybrid operation is required to move so as to perform an appropriate surgery according to a surgical method at the surgery position. Thus, it is preferable that the robot arm be designed such that the vertical position of the table top can be lowered at least to 70 cm and preferably to 50 cm from the floor surface, and increased at least to 100 cm and preferably to 120 cm from the floor surface. For example, in the case in which the table top is moved in the vertical direction while staying parallel to the horizontal plane as illustrated in FIG. 12, the distance H1 from the floor surface to the top surface of the table top when the table top is located at the lowermost position is 50 cm or more and 70 cm or less, and the distance H2 from the floor surface to the top surface of the table top when the table top is located at the uppermost position is 100 cm or more and 120 cm or less.

In the above-described hybrid operation, only one medical imaging device (i.e., a modality) is used in combination with the robotic table. However, a plurality of medical imaging devices may be used in combination with the robotic table. In such a case, positioning of the medical imaging devices may be determined based on the same basis as described above. It is preferable, however, to redesign the medical system in consideration of the positional relationship among the medical imaging devices, and where to install the anesthesia machine, for example.

[Process Management System]

(General Description of System)

FIG. 26 is a block diagram illustrating a configuration for controlling a process management system 700. In the process management system 700, processes of medical practice performed on the target placed on the table top are managed by an integrated controller 701.

The integrated controller 701 is connected to a process display 702, a notifying device 703, an operating device 705, and a robot arm controller 207 to 2907.

The integrated controller 701 is comprised, for example, of an electronic calculator, such as a computer like a central processing unit (CPU). The integrated controller 701 includes a memory 704 comprised, for example, of a hard disk drive (HDD) and a solid state drive (SSD).

Medical processes are input through the operating device 705. Medical processes may be stored in advance so as to be able to select a medical process candidate. Alternatively, processes needed for each medical institution may be made, which may be given names and input. A series of data relating to the medical processes and input through the operating device 705 are stored in the memory 704. Once the process management system 700 is actuated, the series of data relating to the medical processes and stored in the memory 704 are read by the integrated controller 701. In the case in which a plurality of series of data are stored, the medical process data to be used may be selected by the operating device 705 when the data is read.

The operating device 705 includes: an insertion instruction section 715 which gives a command to start a process insertion operation; a process selection section 725 which gives a command to select one from a plurality of processes stored in the memory 704; a deletion instruction section 735 which gives a command to start a process deletion operation; a process designating section 745 which gives a command to designate a process to be inserted, replaced, and deleted; a correction instruction section 755 which gives a command to start a process replacement operation; an enter/complete instruction section 775 which enters the designation or selection of each process or gives a command to complete the current process; a stop instruction section 785 which gives a command to stop movements of the robot arm 201 to 2901; and a progress instruction section 795 which gives a command to move on to the next process from the current process.

The integrated controller 701 reads the medical processes stored in the memory 704, and sets the robot arm 201 to 2901 supporting the table top 208 to 2908 to be in a position or posture corresponding to each process in response to a command from the operating device 705. The position or posture of the robot arm 201 to 2901 do not have to be decided to be the only one position or posture, as long as the position or posture of the robot arm achieves the intended position of the table top 208 to 2908 in the plan of the medical room (the position of the table top when viewed from vertically above) for each process. Setting of the position of the table top 208 to 2908 for each process by the integrated controller 701 in the plan of the medical room will be described later.

The process display 702 is a portion where the medical processes are displayed in a sequential manner, based on the series of data relating to the medical processes read by the integrated controller 701 from the memory 704. The process display 702 is comprised, for example, of a display monitor. The process display 702 includes a current process display section 712 which shows the current progress among the overall medical processes, and a movement display section 722 which displays movements of the table top 208 to 2908 in conjunction with the movements of the robot arm 201 to 2901. The notifying device 703 is comprised of an audio generator such as a speaker, an image display device such as a display, or a light emitting device such as an LED, or a combination of these devices. The notifying device 703 is controlled by the integrated controller 701, and provides information relating to the movements of the robot arm 201 to 2901.

(Process Progress Management in Intraoperative MRI for Brain Tumor Removal)

Brain tumor removal surgery using intraoperative MRI will be described as example medical processes for progress management. In general, a brain image is taken by MRI before brain tumor removal surgery; the patient undergoes craniotomy; and the surgery is performed while viewing the brain image taken beforehand. After the removal of the tumor, a brain image is taken again by MRI to check the degree of removal of the tumor. If the tumor is thoroughly removed, the process proceeds to a surgery end process, and if not, the tumor removal surgery is continued.

Overall processes of such intraoperative MRI include, for example: (1) a waiting process; (2) a placement process; (3) an anesthesia induction process; (4) a pre-surgery preparation process; (5) a surgery process; (6) an MRI process; (7) a post-surgery treatment process; (8) an anesthesia recovery process; and (9) an ending process, which are scheduled and stored in advance.

(1) The waiting process is a process until a patient is placed on the table top 208 to 2908. In the waiting process, a drip and the arrangements of equipment around the operating table are checked. When the patient K enters the operating room, it is reaffirmed that the patient K is the operation subject; the food intake condition and physical condition are checked; explanation about the surgery is made, and so on. The table top may be located at a basic position, where the robot arm is in basic position and posture as illustrated in FIGS. 6 and 13, or at the same position as the anesthesia induction position illustrated in FIGS. 22 and 23, or at any other positions.

(2) The placement process is a process in which the patient walks to and lies on the table top 208 to 2908, or is relocated from a stretcher onto the table top 208 to 2908. An assistant confirms secure placement of the patient and fall prevention measures (e.g., attachment of a belt holding the patient), and tells the patient that the table top 208 to 2908 will be moving for anesthesia induction.

(3) The anesthesia induction process is a process in which the patient undergoes anesthesia. In the anesthesia induction process, an oxygen mask is applied to the patient, and an anesthetic agent is administered to the patient through a drip or an artificial respiration tube, while telling the patient, if necessary, that the patient is gradually losing consciousness.

(4) The pre-surgery preparation process is a process in which medical instruments necessary for the surgery are connected, and medications necessary for maintaining the physical condition of the patient are administered. For example, the process includes connecting a brain wave monitor, a transfusion pump, a patient's temperature keeping device, and so on. The process further includes shaving head, disinfecting the surgery site, draping, and so on.

(5) The surgery process is a process in which a surgeon performs surgery to remove a brain tumor while viewing the brain image taken in advance, using a navigation system or the like if necessary. First, the assistant makes an incision in the head skin and separates a bone by using a drill or the like. The surgeon then removes the brain tumor by using a surgical instrument, such as a raspatory.

(6) The MRI process is a process in which the patient's head is subjected to imaging by an MRI apparatus to obtain a brain image. The surgeon checks the obtained brain image to see the degree of removal of the tumor, and determines whether to continue or end the surgery.

(7) The post-surgery treatment process is a process for restoring the surgery site to its original state as much as possible after the tumor removal. In this process, the assistant cleans the surgery site, and the head is closed. Also in this process, the drape is removed and the medical equipment and transfusion pump connected to the patient are detached.

(8) The anesthesia recovery process is a process for ending the anesthesia for the patient and bringing the patient back to consciousness. In this process, an anesthesiologist operates the anesthesia machine to bring the patient back to consciousness.

(9) The ending process is a remaining process until the patient leaves the operating room. In this process, after the patient comes back to consciousness, the patient is placed on a stretcher and carried out of the operating room.

In the series of processes of the brain tumor removal surgery, the robotic table having the above-described example configurations is used as the robotic operation table, and the position of the table top 208 to 2908 in the plan of the medical room is stored so as to correspond to each process. The relationship between each process and the position of the table top 208 to 2908 is as follows, for example.

(1) In the waiting process, the table top 208 to 2908 is located at the basic position illustrated, for example, in FIGS. 6, 13, and 19. The table top is arranged at the lowest height, and the robot arm takes a posture which does not apply load to the brakes and other elements of the robot arm even when the power is turned off.

(2) In the placement process, the table top 208 to 2908 is located, for example, at the anesthesia induction position illustrated in FIGS. 22 and 23. The table top is arranged at a low height so that the patient can be laid on the table top easily. The suitable height of the table top differs depending on the patient's height, whether the patient walks into the operating room and lies on the table top, or the patient is carried into the operating room on a stretcher and relocated on the table top. The height of the table top is therefore appropriately adjusted through the operating device (it may be enough to store only the position of the table top in the plan of the medical room). Note that the placement position may be different from the anesthesia induction position, and may be the same as a waiting position (the basic position).

(3) In the anesthesia induction process, the table top 208 to 2908 is set at the anesthesia induction position illustrated in FIGS. 22 and 23. In head surgery, such as the brain tumor removal surgery described above, an area around the head is set to be a clean area. Thus, the anesthesia machine is preferably located at a side of the table top opposite to the patient's head. On the other hand, in cardiac surgery, for example, an area around the head is set to be an unclean area. Thus, where to position the anesthesia machine and the table top is also different. The table top is arranged at a height that allows easy anesthesia induction, according to the height of the anesthesia machine.

(4) In the pre-surgery preparation process, the table top may be basically located at the same position as the surgery position. The table top position may be the same as, or different from, the basic position illustrated in FIGS. 6, 13, and 19.

(5) The table top 208 to 2908 in the surgery process is basically set to be the same position as the table top position in the pre-surgery preparation process, but may be set to be a different position farther away from the medical equipment, compared to the table top position in the pre-surgery preparation process. The table top position in the surgery process is determined so that there is not much medical equipment around the table top and therefore that a lot of team members (e.g., a surgeon, an assistant, and a nurse) can surround the table top, and so that the table top is located at a position at least a predetermined distance from the MRI for safety.

(6) In the MRI imaging process, the table top 208 to 2908 is located at the imaging position illustrated, for example, in FIGS. 8, 15, and 21. In the case of the table top with a man-powered slide, the table top position is the imaging preparation position illustrated in FIG. 20.

(7) In the post-surgery treatment process, the table top 208 to 2908 is located, for example, at the surgery position or a pre-surgery preparation position.

(8) In the anesthesia recovery process, the table top 208 to 2908 is located, for example, at the same position as the anesthesia induction position. The table top position is preferably the same as the anesthesia induction position because the anesthesiologist controls the anesthesia machine for patient's recovery from anesthesia. The table top is arranged at a height that allows easy operation of the anesthesia machine, according to the height of the anesthesia machine.

(9) In the ending process, the table top 208 to 2908 is located, for example, at the same position as the anesthesia induction position. The patient is generally carried out from the operating room on a stretcher after the surgery. Thus, the table top is arranged at a height that allows easy relocation of the patient to the stretcher, according to the height of the stretcher.

(Process Progress Management in Hybrid Operation for Aneurysm Clipping)

Aneurysm neck clipping surgery in the hybrid operation using an angiographic device will be described as different example medical processes for progress management. In general, in the aneurysm neck clipping surgery, too, a brain image is taken by the angiographic device before the surgery; the patient undergoes craniotomy; and the surgery is performed while viewing the brain image taken beforehand. After the clipping, a brain image is taken again by the angiographic device to check the state of the blood vessels after the clipping according to necessity. If the state of the blood vessels is good, the process proceeds to a surgery end process, and if not, the clipping is performed again.

Overall processes of such a hybrid operation include, for example: (1) a waiting process; (2) a placement process; (3) an anesthesia induction process; (4) a pre-surgery preparation process; (5) a surgery process; (6) an angiographic imaging process; (7) a post-surgery treatment process; (8) an anesthesia recovery process; and (9) an ending process, which are scheduled and stored in advance.

(1) The waiting process is a process until a patient is placed on the table top 208 to 2908. In the waiting process, a drip and the arrangements of equipment around the operating table top are checked. When the patient enters the operating room, it is reaffirmed that the patient is the operation subject; the food intake condition and physical condition are checked; explanation about the surgery is made, and so on. The table top may be located at a basic position, where the robot arm 201 to 2901 is in basic position and posture as illustrated in FIGS. 6 and 13, or at the same position as the anesthesia induction position illustrated in FIGS. 22 and 23, or at any other positions.

(2) The placement process is a process in which the patient walks to and lies on the table top 208 to 2908, or is relocated from a stretcher onto the table top 208 to 2908. An assistant confirms secure placement of the patient and fall prevention measures (e.g., attachment of a belt holding the patient), and tells the patient that the table top 208 to 2908 will be moving for anesthesia induction.

(3) The anesthesia induction process is a process in which the patient undergoes anesthesia. In the anesthesia induction process, an oxygen mask is applied to the patient, and an anesthetic agent is administered to the patient through a drip or an artificial respiration tube, while telling the patient, if necessary, that the patient is gradually losing consciousness.

(4) The pre-surgery preparation process is a process in which medical instruments necessary for the surgery are connected, and medications necessary for maintaining the physical condition of the patient are administered. For example, the process includes securing the patient's head with a head rest to prevent movement of the patient's head, and connecting a brain wave monitor, a transfusion pump, a patient's temperature keeping device, and so on. The process further includes shaving head, disinfecting the surgery site, draping, and so on. Further, the table top is bent to change the posture of the patient to a posture easy to perform surgery. The table top for use in the aneurysm neck clipping surgery is bendable in the longitudinal direction of the table top.

(5) The surgery process is a process in which a surgeon performs surgery while viewing the brain image taken in advance, using a navigation system or the like if necessary. First, the assistant makes an incision in the head skin and separates a bone by using a drill or the like, and the surgeon takes over thereafter. Using a surgical microscope, the surgeon makes access to the aneurysm and performs clipping using a surgical instrument, such as a bipolar.

(6) The angiographic imaging process is a process in which the table top 208 to 2908 is returned to a flat state and the patient's head is subjected to imaging by the angiographic device to obtain a brain image. The surgeon checks the obtained brain image to see the state of the blood vessels, and determines whether to continue or end the surgery.

(7) The post-surgery treatment process is a process for restoring the surgery site to its original state as much as possible after the completion of the aneurysm clipping. In this process, the assistant cleans the surgery site, and the head is closed. Also in this process, the drape is removed and the medical equipment and transfusion pump connected to the patient are detached.

(8) The anesthesia recovery process is a process for ending the anesthesia for the patient and bringing the patient back to consciousness. In this process, an anesthesiologist operates the anesthesia machine to bring the patient back to consciousness.

(9) The ending process is a remaining process until the patient leaves the operating room. In this process, after the patient comes back to consciousness, the patient is placed on a stretcher and carried out of the operating room.

These are a series of medical processes of the aneurysm neck clipping surgery. In the series of processes, the robotic table having the above-described example configurations is used as the robotic operation table, and the position of the table top is stored so as to correspond to each process. The relationship between each process and the position of the table top is as follows, for example.

(1) In the waiting process, the table top 208 to 2908 is located at the basic position illustrated, for example, in FIGS. 6, 13, and 24A and 24B. The table top is arranged at the lowest height, and the robot arm takes a posture which does not apply load to the brakes and other elements of the robot arm even when the power is turned off.

(2) In the placement process, the table top 208 to 2908 is located at the anesthesia induction position illustrated in FIGS. 22 and 23. The table top is arranged at a low height so that the patient can be laid on the table top easily. The suitable height of the table top differs depending on the patient's height, whether the patient walks into the operating room and lies on the table top, or the patient is carried into the operating room on a stretcher and relocated on the table top. The height of the table top is therefore appropriately adjusted manually through the operating device (it may be enough to store only the position of the table top in plan of the medical room). Note that the placement position may be different from the anesthesia induction position, and may be the same as a waiting position (the basic position).

(3) In the anesthesia induction process, the table top 208 to 2908 is set at the anesthesia induction position illustrated in FIGS. 22 and 23. In head surgery, such as the aneurysm neck clipping surgery described above, an area around the head is set to be a clean area. Thus, the anesthesia machine is preferably located at a side of the table top opposite to the patient's head.

(4) In the pre-surgery preparation process, the table top may be basically located at the same position as the surgery position. The table top position may be the same as, or different from, the basic position illustrated in FIGS. 6, 13, and 24A and 24B.

(5) The table top 208 to 2908 in the surgery process is basically set to be the same position as the table top position in the pre-surgery preparation process, but may be set to be a different position farther away from the medical equipment, compared to the table top position in the pre-surgery preparation process. The table top position in the surgery process is determined so that a lot of team members (e.g., a surgeon, an assistant, and a nurse) can surround the table top, and that the surgical microscope can be located near the patient's head.

(6) In the angiographic imaging process, the table top 208 to 2908 is located at the imaging position illustrated, for example, in FIGS. 8, 15, and 24A and 24B. In the case of the table top with a man-powered slide, the table top position is the imaging preparation position illustrated in FIGS. 24C and 24D.

(7) In the post-surgery treatment process, the table top 208 to 2908 is located, for example, at the surgery position or a pre-surgery preparation position.

(8) In the anesthesia recovery process, the table top 208 to 2908 is located, for example, at the same position as the anesthesia induction position. The table top position is preferably the same as the anesthesia induction position because the anesthesiologist controls the anesthesia machine for patient's recovery from anesthesia. The table top is arranged at a height that allows easy operation of the anesthesia machine, according to the height of the anesthesia machine.

(9) In the ending process, the table top 208 to 2908 is located, for example, at the same position as the anesthesia induction position. The patient is generally carried out from the operating room on a stretcher after the surgery. Thus, the table top is arranged at a height that allows easy relocation of the patient to the stretcher, according to the height of the stretcher.

(Specific Configuration of System)

The integrated controller 701 stores, for example, table top position data (e.g., coordinate data) for each of the above nine processes in the memory 704. The integrated controller 701 transmits a control instruction signal to each robot arm controller 207 to 2907 so that the position and posture of the table top correspond to those of the table top in the plan of the medical room.

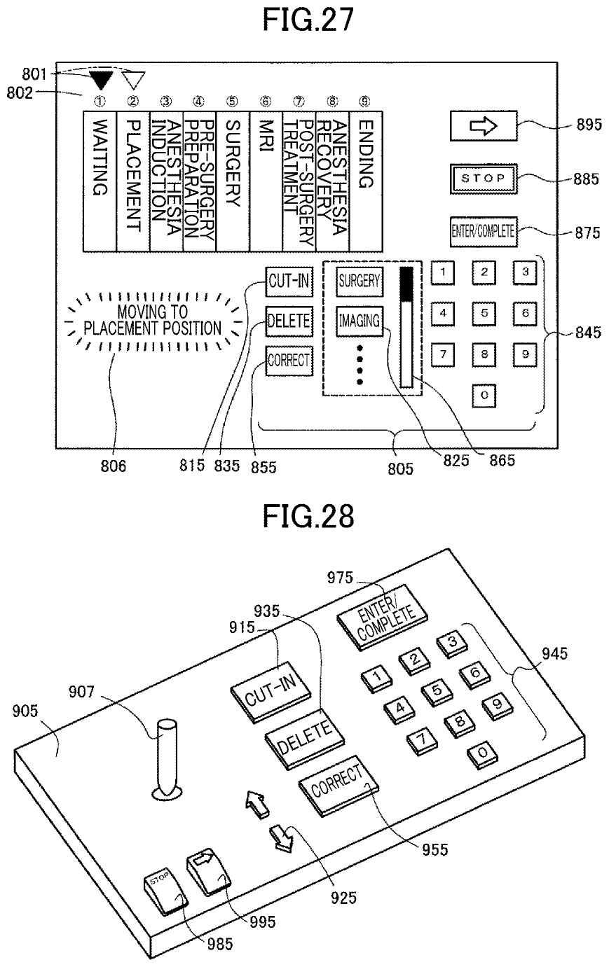

FIG. 27 is a diagram of a monitor as the process display 702. A series of data relating to the medical processes stored in the memory 704 are read by the control of the integrated controller 701, and a series of medical processes are displayed on the monitor in a scheduled process order with associated process numbers. A large monitor, e.g., a 50 inch monitor, is preferable so that the whole team can share the information. Displaying the overall processes in a list in the above-described manner allows the whole team to acquire the total time required per surgery and the progress of the surgery. The display illustrated in FIG. 27 may be configured such that the width of the frame representing each process is variable according to the expected time required for the process. This configuration makes it possible to acquire the proportion of each process to the overall processes.

Among the series of processes displayed, the current process is indicated by a cursor 801, which is the current process display section 712, through the control of the integrated controller 701. The current process display section 712 is not limited to the cursor 801, and may include, for example, reversing colors of the background and text (e.g., black background and white text) or increasing the border thickness of the current process to differentiate the current process from the other processes.

The monitor 802 illustrated in FIG. 27, which functions as the process display 702, is capable of a touch panel operation, and functions also as the operating device 705. An operation button display area 805 includes: a cut-in button display 815 functioning as the insertion instruction section 715; a process candidate display 825 functioning as the process selection section 725; a scrollbar display 865 for scrolling and showing the process candidates in the process candidate display 825; a deletion button display 835 functioning as the deletion instruction section 735; a ten-key display 845 functioning as the process designating section 745; a correction button display 855 functioning as the correction instruction section 755; an enter/complete button display 875 functioning as the enter/complete instruction section 775; a stop button display 885 functioning as the stop instruction section 785; and a right-arrow button display 895 function as the progress instruction section 795.

The instruction signal is output to the integrated controller 701 by touching a corresponding button display displayed in the operation button display area 805.

The operating device 705 is not limited to a touch panel monitor, and may be a computer equipped with a keyboard and a mouse and independently provided from the monitor 802, or a user interface operated with buttons as illustrated in FIG. 28. The operating device 705 may also be a teaching pendant as mentioned in each example configuration of the robotic table.

The operating device 905 illustrated in FIG. 28 has a plurality of manually-operated push buttons. Such buttons include: a cut-in button 915 functioning as the insertion instruction section 715; up and down arrow buttons 925 functioning as the process selection section 725 (the process to be selected is displayed on the process display 702); a deletion button 935 functioning as the deletion instruction section 735; a ten-key 945 functioning as the process designating section 745; a correction button 955 functioning as the correction instruction section 755; an enter/complete button 975 as the enter/complete instruction section 775; a stop button 985 functioning as the stop instruction section 785; and a right-arrow button 995 functioning as the progress instruction section 795. The instruction signal is output to the integrated controller 701 by operating these buttons.

The right-arrow button 895 and 995 is operated to move the cursor 801, which is the current process display section 712 displayed on the monitor 802, to the process on the right of the current process (in FIG. 27, to the placement process indicated by the imaginary two-dot chain line). The integrated controller 701 transmits a command to the robot arm controller 207 to 2907 so that the position and posture of the robot arm 201 to 2901 be set to the position of the table top 208 to 2908 for the next process. For the safety purpose, the robot arm 201 to 2901 is allowed to move only while the right-arrow button 895 and 995 is being operated (i.e., being pressed). The robot arm 201 to 2901 may automatically move to the position and posture corresponding to the next process by one operation of the right-arrow button 895 and 995, in which the robot arm automatically moves until the robot arm reaches the target position, unless receiving a command from the stop instruction section 785.

When the table top 208 to 2908 reaches the respective target position by the control of the operating device 705, the electromagnetic brake of each joint of the robot arm 201 to 2901 is activated automatically, and the position and posture of the robot arm 201 to 2901 are fixed. Alternatively, movements of the table top 208 to 2908 are stopped even while the operating device 705 is being operated (for example, even while the right-arrow button 995 is being pressed), and the electromagnetic brake is activated at the time when the operation of the operating device 705 is stopped (for example, when the right-arrow button 995 is released from pressing), so that the position and posture of the robot arm 201 to 2901 are fixed.

In the above described one or more examples, the monitor 802 includes the touch panel area functioning as the operating device 705. However, the operating device 705 may be the operating device 905 illustrated in FIG. 28 with a compact display and a speaker functioning as the process display 702 and the notifying device 703, respectively.

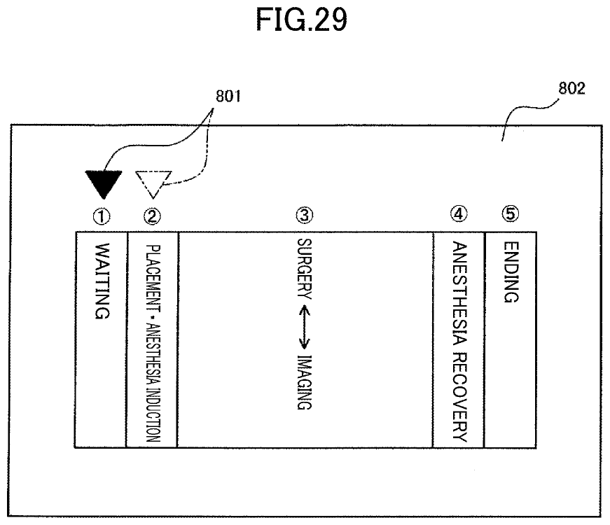

Not every process in the series of medical processes may be completed by performing once. For example, the medical processes may include a process in which the surgery process and the imaging process are repeated multiple times. In the medical processes illustrated in FIG. 29, the “surgery and imaging” process includes alternately repeating the surgery process and the imaging process based on commands from the progress instruction section 795. The “surgery and imaging” process is completed by the command from the enter/complete instruction section 775, and proceeds to the next anesthesia recovery process by a subsequent command from the progress instruction section 795.

If, for example, the placement position and anesthesia induction position of the table top 208 to 2908 are the same, the two processes may be combined into a “placement and anesthesia induction process.” Alternatively, the placement process may be deleted and the process may be referred to as the anesthesia induction process. Such creation and change of the medical processes can also be accomplished by the operating apparatus 705.

The operating device 705 may have a joystick 907 as illustrated in FIG. 28. For example, a progress instruction signal for causing the process to proceed is input to the integrated controller 701 by an operator leaning the joystick rightward. A stop instruction signal for stopping, for example, the movements of the robot arm 201 to 2901 is input to the integrated controller 701 by leaning the joystick 907 leftward.

The monitor 802 illustrated in FIG. 27 includes a movement display area 806 as the movement display section 722. The movement display area 806 displays, for example, the following states of movements of the robotic operation table, in response to the commands from the progress instruction section 795 and by the control of the integrated controller 701: “Move to Placement Position” right before the table top moves to the placement position (a display indicating that the process is going to move to the process subsequent to the current process); “Moving to Placement Position” while the table top moves to the placement position (a display indicating that the process is moving on to the process subsequent to the current process); “Stopping at Anesthesia Induction Position” during induction of anesthesia; “At Surgery position. Movement Prohibited Till Surgery Completion” while the robot arm 201 to 2901 at the surgery position is completely locked by applying brakes (a display indicating that the movement of the robot arm has been ended). In this manner, the whole team can confirm and share the movement of the robotic operation table and the current process, which can promote the safety in the medical room where the robot arm is installed.

The process management system described herein also provides voice notification about the state of movement of the robot arm 201 to 2901 through the notifying device 703, in conjunction with the movement of the robot arm 201 to 2901 and by the control of the integrated controller 701. The notifying device 703 is, for example, a speaker installed in the medical room. For example, the notifying device 703 may provide voice notification, such as “Move to placement position” throughout the medical room, right before the table top 208 to 2908 moves to the placement position from the waiting position (to notify before movement of the robot arm that the robot arm is going to move), “Moving to surgery position” throughout the medical room, while the table top 208 to 2908 is moving to the surgery position from the anesthesia induction position (to notify during the movement of the robot arm that the robot arm is moving), “Arrived at surgery position. Brakes have been applied and the movements have been completely stopped” throughout the medical room when the robotic table has arrived at the surgery position and has been completely locked (to notify that the movement of the robot arm has been ended). In this manner, the whole team can confirm and share the movement of the robotic table and the current process through the voice notification as well, which can promote the safety in the medical room where the robot arm is installed. The display of the state of movements and the voice notification may also be used in combination.

(Application to Other Medical Processes)

The above medical control system is applicable not only to the illustrated hybrid operation, but also to medical processes such as treatment and inspection processes, as long as the medical processes utilize a robotic table.

For example, the system is also applicable to the case in which a robotic table is transferred to a medical imaging device just to take an image.

A process management system for use at medical settings using robotic tables of the first to third example configurations has been described. However, one or more embodiments disclosed herein may be modified in various manners without departing from the scope of the invention. For example, the robotic table is not limited to the robotic tables of the first to third example configurations, and the shape of the robot arm is also not limited to the shapes illustrated herein. The shape of the table top is not limited to a rectangle in each of the drawings, and the table top may be bendable so that the table top can take various postures.

The process management system having the above configurations allows the robotic tables of the respective example configurations to be used more efficiently, and can improve the utilization rate of a medical room.

1. A medical system, comprising:
a memory which stores data relating to medical processes including at least a placement process, an imaging process, and a surgery process;
a robotic operation table including a table top on which a patient is to be placed and a robot arm supporting the table top, wherein the robot arm includes: a plurality of movable elements; a plurality of joints connecting the plurality of movable elements to one another; a plurality of electric actuators provided corresponding to the plurality of joints respectively; and a plurality of position detectors provided corresponding to the plurality of joints respectively; and wherein at least one of the plurality of joints is a horizontally-rotating joint which couples two movable elements among the plurality of movable elements to each other so as to be rotatable about a vertical axis; and
a controller which gives the robot arm a command, which is based on the data relating to the medical processes stored in the memory, to move the table top to positions corresponding to the medical processes including a placement position where the patient is placed on the table top, an imaging position where an image of the patient is taken by a medical imaging device, and a surgery position where surgery is performed on the patient.

2. The medical system of claim 1, wherein
the data relating to the medical processes is stored in the memory such that the medical processes including at least the placement process, the imaging process, and the surgery process are arranged in a sequential manner.

3. The medical system of claim 1, further comprising a monitor, wherein
the controller causes the monitor to display the data relating to the medical processes.

4. The medical system of claim 3, wherein
the controller causes the monitor to display a current process among the medical processes.

5. The medical system of claim 4, wherein
the controller causes the monitor to display, before the robot arm moves, an indication that the current process will be terminated to transition to a next process.

6. The medical system of claim 4, wherein
the controller causes the monitor to display, while the robot arm is moving, an indication that the current process is terminating to transition to a next process.

7. The medical system of claim 3, wherein
the controller causes the monitor to display, after completion of the movement of the robot arm, an indication that the movement of the robot arm has been ended.

8. The medical system of claim 1, further comprising a speaker, wherein
the controller causes the speaker to provide voice notification about a state of movement of the robot arm.

9. The medical system of claim 8, wherein
the controller causes the speaker to provide voice notification to notify, before the robot arm moves, that the robot arm will be moving.

10. The medical system of claim 8, wherein
the controller causes the speaker to provide voice notification to notify, while the robot arm is moving, that the robot arm is moving.

11. The medical system of claim 8, wherein
the controller causes the speaker to provide voice notification to notify, after completion of the movement of the robot arm, that the movement of the robot arm has been ended.

12. The medical system of claim 1, wherein
the controller causes the robot arm to move in response to an instruction from an operating device.

13. The medical system of claim 2, further comprises
an operating device includes an insertion instruction section and a process selection section, wherein
the process selection section is configured to receive a user input to select one of candidate medical processes stored in the memory, and
the insertion instruction section is configured to receive a user input to add or insert the selected process selected by the process selection section to the sequential medical processes.

14. The medical system of claim 2, further comprising
an operating device includes a deletion instruction section and a process designating section,
the process designating section is configured to receive a user input to designate one from the sequential medical processes to be deleted, and
the deletion instruction section is configured to receive a user input to delete the designated process designated by the process designating section from the sequential medical processes.

15. The medical system of claim 2, further comprising
an operating device includes a correction instruction section, a process designating section, and a process selection section,
the process selection section is configured to receive a user input to select one from candidate medical processes stored in the memory,
the process designating section is configured to receive a user input to designate one from the sequential medical processes to be replaced, and
the correction instruction section is configured to receive a user input to replace the designated process designated by the process designating section with the selected process selected by the process selection section in the sequential medical processes.

16. The medical system of claim 1, wherein
the medical processes are processes of a hybrid operation or processes of intraoperative a magnetic resonance imaging.

17. The medical system of claim 1, wherein
the medical processes include an anesthesia induction process in which anesthesia is administered to the patient, and
the positions corresponding to the medical processes further include an anesthesia induction position where the patient is anesthetized using an anesthesia machine.

18. A table top transfer method executed by a controller in a medical system which comprises a memory which stores data relating to medical processes including at least a placement process, an imaging process, and a surgery process, and a robotic operation table including a table top on which a patient is to be placed and a robot arm supporting the table top,
the method comprising:
positioning the table top, by the robot arm, at a placement position where the patient is placed on the table top, by reading the data relating to the placement process from the memory and giving a command to the robot arm;
positioning the table top, by the robot arm, at a surgery position where surgery is performed on the patient, by reading the data relating to the surgery process from the memory and giving a command to the robot arm; and
positioning the table top, by the robot arm, at an imaging position where an image of the patient is taken by a medical imaging device, by reading the data relating to the imaging process from the memory and giving a command to the robot arm,
wherein the robot arm comprises a plurality of movable elements, and a plurality of joints connecting the plurality of movable elements, a plurality of electric actuators provided corresponding to the plurality of joints respectively, and a plurality of position detectors provided corresponding to the plurality of joints respectively, wherein at least one of the plurality of joints is a horizontally-rotating joint which couples two movable elements among the plurality of movable elements to each other so as to be rotatable about a vertical axis.

19. The table top transfer method of claim 18, wherein
the positioning of the table top at the surgery position and the positioning of the table top at the imaging position are repeated two or more times.

20. A robotic operation table comprising:
a table top on which a patient is to be placed; and
a robot arm supporting the table top, the robot arm comprising a plurality of movable elements, a plurality of joints connecting the plurality of movable elements, a plurality of electric actuators provided corresponding to the plurality of joints respectively, and a plurality of position detectors provided corresponding to the plurality of joints respectively, wherein at least one of the plurality of joints is a horizontally-rotating joint which couples two movable elements among the plurality of movable elements to each other so as to be rotatable about a vertical axis, wherein
the robot arm is configured, based on data relating to medical processes including at least a placement process, an imaging process, and a surgery process stored in a memory, to position the table top to positions corresponding to the medical processes including a placement position where the patient is placed on the table top, an imaging position where an image of the patient is taken by a medical imaging device, and a surgery position where surgery is performed on the patient.
